(19) United States
(12) Patent Application Publication
(10) Pub. No.: US 2013/0324555 A1
(43) Pub. Date: Dec. 5, 2013

(54) Method of use of pharmaceutical formulations for the treatment of apicomplexan diseases in animals

(71) Applicants: Richard Delarey Wood, Washington Crossing, PA (US); Rima McLeod, Chicago, IL (US); Ernest Mui, Glasgow (GB); Alina Fomovska, New York, NY (US); William Welsh, Princeton, NJ (US)

(72) Inventors: Richard Delarey Wood, Washington Crossing, PA (US); Rima McLeod, Chicago, IL (US); Ernest Mui, Glasgow (GB); Alina Fomovska, New York, NY (US); William Welsh, Princeton, NJ (US)

(21) Appl. No.: 13/904,063

(22) Filed: May 29, 2013

Related U.S. Application Data
(60) Provisional application No. 61/689,066, filed on May 29, 2012.

(51) Int. Cl. C07C 235/64; C07C 271/50; C07D 295/192

(52) U.S. Cl. CPC C07C 235/64; C07D 295/192; C07C 271/50
USPC 514/255.01; 514/622; 514/534; 514/490

(57) ABSTRACT

The present invention is directed to the method of use of effective pharmaceutical formulations for the treatment of diseases caused by apicomplexan parasites, said formulation comprised of a salicylanilide or salicylanilide derivative, disclosed herein, alone or in combination with one or more other active or excipient pharmaceutical substances. The present invention is further directed to the method of use of effective pharmaceutical formulations for the treatment of diseases caused by apicomplexan parasites, said formulation comprised of a combination of salicylanilides or salicylanilide derivatives, disclosed herein. The present invention is further directed to the method of use of effective pharmaceutical formulations for the treatment of diseases caused by apicomplexan parasites, said formulation comprised of a combination of salicylanilides or salicylanilide derivatives, disclosed herein, further comprised of one or more active or excipient pharmaceutical substances.

### CROSS-REFERENCE TO RELATED APPLICATIONS

Not Applicable

### BACKGROUND OF THE INVENTION

1. Field of the Invention

The invention relates to the preparation and use of pharmaceutical formulations for the treatment of apicomplexan diseases in animals.

2. Description of the Related Art

Salicylanilides and their derivatives are well-studied compounds. Salicylanilides have been shown to possess a wide range of biological activities, including anti-infective activity. The antimicrobial activity of this class of compounds has been reported (Kratky, M.; Vinsova, J., Current Pharmaceutical Design (2011), 17(32), 3494-3505). In addition, salicylanilides have been shown to possess antiviral activity (Kratky, M.; Vinsova, J., Mini-Reviews in Medicinal Chemistry (2011), 11(11), 956-967). The anti-helmintic activity has been thoroughly studied (Agrawal, V. K.; Sharma, S.; Phormazie (1984), 39(6), 373-8), and the fasciolicidic (Fairweather, I.; Boray, J. C.; Veterinary Journal (1999), 158(2), 81-112), anticestodal, and antineotodal (Lanusse, Carlos E.; Virkel, Guillermo L; Alvarez, Luis I.; Edited by Riviere, Jim Edmond; Papich, Mark G; Veterinary Pharmacology and Therapeutics (9th Edition) (2009), 1095-1117) properties of salicylanilides have been disclosed.

The present invention discloses the anti-apicomplexan activity of certain salicylanilides and salicylanilide derivatives, an important biological activity which has not been previously recognized. The phylum Apicomplexa is composed a myriad of currently recognized species. Of these, several species are medically important and are the causative agents of diseases including, but not limited to, malaria, babesiosis, cryptosporidiosis, cyclosporiasis, isosporiasis, and toxoplasmosis. These insidious diseases are rampant worldwide and cause vast morbidity and mortality. To date, anti-apicomplexan therapies have demonstrated less than optimal efficacy and frequently impart side effect profile which render them inappropriate for use or cause low patient compliance. The currently favored therapeutic intervention for the treatment for toxoplasmosis is the combination cocktail of pyrimethamine and sulfadiazine which frequently causes intolerable side effects. There is currently an unmet need for safe and effective anti-apicomplexan treatments. The present invention is directed to one solution of that unmet need.

### BRIEF SUMMARY OF THE INVENTION

The present invention is directed to the method of use of effective pharmaceutical formulations for the treatment of diseases caused by apicomplexan parasites, said formulation comprised of a salicylanilide or salicylanilide derivative, disclosed herein, alone or in combination with one or more other active or excipient pharmaceutical substances. The present invention is further directed to the method of use of effective pharmaceutical formulations for the treatment of diseases caused by apicomplexan parasites, said formulation comprised of a combination of salicylanilides or salicylanilide derivatives, disclosed herein. The present invention is further directed to the method of use of effective pharmaceutical formulations for the treatment of diseases caused by apicomplexan parasites, said formulation comprised of a combination of salicylanilides or salicylanilide derivatives, disclosed herein, further comprised of one or more active or excipient pharmaceutical substances.

### BRIEF DESCRIPTION OF THE DRAWINGS

FIG. 1 portrays the inhibition of T. gondii RH-YFP fluorescence in the presence of Compound 3i.

FIG. 2 depicts the inhibition of T. gondii RH-YFP fluorescence in the presence of Compound 3j.

FIG. 3 portrays the inhibition of T. gondii RH-YFP fluorescence in the presence of Compound 7a.

FIG. 4 portrays the inhibition of T. gondii RH-YFP fluorescence in the presence of Compound 14a.

FIG. 5 shows the inhibition of T. gondii RH-YFP fluorescence in the presence of Compound 14b.

FIG. 6 displays the effect of selected compounds on survival of HFF Cells.

FIG. 7 depicts the effect of various dosing conditions on prolonged survival of RH-YFP tachyzoites, measured by inhibition of RH-YFP fluorescence.

FIG. 8 portrays the effect of Compound 14a on survival in a mouse model of T. gondii Me-49 oocysts.

FIG. 9 portrays the effect of Compound 14b on survival in a mouse model of T. gondii TgGoatUS4 oocysts.

### DETAILED DESCRIPTION OF THE INVENTION

The Apicomplexan family of parasites, which includes members such as Plasmodia, Babesia and Toxoplasma, are protozoa of great medical and economic significance. T. gondii is one of the most successful parasites on earth, infecting all warm-blooded animals and one-third to one-half of the human population. This parasite can cause disease, toxoplasmosis, with eye and neurological damage, systemic illness, and death. Toxoplasmosis can be especially devastating in those infected congenitally or immune-compromised persons or those with post-natal acquired infection.

Though the only definitive host of this obligate, intracellular parasite are members of the Felidae (cat) family, in people and other intermediate hosts T. gondii exists in two life stages: the rapidly proliferating tachyzoite form, and the latent, encysted bradyzoite form, which remains in the body for the duration of the lifetime of the host, maintaining the risk of recurrence. There are currently no effective treatments against the bradyzoite form, and those medicines which target the tachyzoite form—Pyrimethamine and Sulfadiazine are the most effective—can be associated with toxicity and hypersensitivity. Novel, nontoxic anti-Toxoplasma agents are greatly needed.

Niclosamide (5-chloro-N-(2-chloro-4-nitrophenyl)-2-hydrobenzamide, 4) is a well-established FDA-approved anti-helmintic drug whose activity in the tapeworm is thought to involve the uncoupling of oxidative phosphorylation. It is not toxic at high concentrations when administered orally. Earlier unpublished studies by our group had identified niclosamide as a potential inhibitor of T. gondii (MIC₅₀250-200 nM). Although niclosamide has the disadvantage of low solubility and low bioavailability, its promising activity against T. gondii inspired the preparation and testing of a series of salicylanilides and derivatives in the hope of potentially improving potency and physicochemical and pharmacological properties. These were evaluated for activity against T. gondii tachyzoites and for toxicity towards host cells in vitro. Experiments were conducted to determine whether the observed activity was due to static or cidal effects. The most promising inhibitors which emerged from this study were the carbamate derivatives 14a and 14b which possess an ionizable moiety appended to the salicylanilide core.

As an Apicomplexan parasite, T. gondii is often used as a model organism to study other members of this family, such as Plasmodium, Babesia and Eimeria. Because the potential of activity of our compounds against other apicomplexans is of great interest, selected compounds were also tested for efficacy against both drug-sensitive and drug-resistant strains of Plasmodium falciparum, the causative agent of malaria, and were found to be effective as described herein. The present invention is directed to preparations comprised of one or more active anti-apicomplexan compounds and their use to prevent or treat apicomplexan infections.

“Active anti-apicomplexan compounds” means those chemical entities disclosed herein which inhibit the growth, motility, invasion, or survival of one or more Phylum Apicomplexa species, wherein the compound possesses the general chemical structure of Formula I, and pharmaceutically acceptable salts, prodrugs, enantiomers, or hydrates thereof:

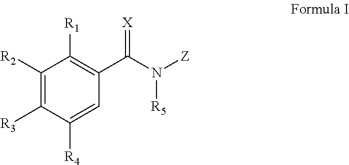

wherein

### R₁═H, SH, or OR₆;

R₂ and R₃ are selected from fused phenyl, H, lower alkyl, I, Br, Cl, F, CF₃, CH₂CF₃, CH₂Ph, CH═CH₂, C≡CH, OCH₃, OCF₃, Ph, OPh, and NO₂;
R₄ is selected from the group consisting of H, lower alkyl, I, Br, Cl, F, CF₃, CH₂CF₃, CH₂Ph, CH═CH₂, C≡CH, C≡N, OCH₃, OCF₃, Ph, OPh, and NO₂;
R₅ is H, lower alkyl, or phenyl;
R₆ is selected from the group consisting of H, COCH₃, COCH₂CH₃, COCH(CH₃)₂, COC(CH₃)₃, COPh, COCH₂Ph, COC₆H₄NO₂(p), COC₆H₄OH(p), COC₆H₄NH₂(p), CON(CH₃)₂, CON(CH₂CH₃)₂, CON(CH₃)(CH₂CH₃), CON(CH₂Ph)₂, CON(CH₃)(CH₂Ph), 1-pyrrolidinecarbonyl, 2-carboxy-1-pyrrolidinecarbonyl, (2S)-2-carboxy-1-pyrrolidinecarbonyl, (2R)-2-carboxy-1-pyrrolidinecarbonyl, 1-morpholinecarbonyl, 4-methyl-1-piperazinecarbonyl, sarcosine-N-carbonyl, CO-N-Me-Ala-OH, CO-N-Me-Val-OH, CO-N-Me-Leu-OH, CO-N-Me-Ile-OH, CO-N-Me-Val-OH, CO-N-Me-Met-OH, CO-N-Me-Phe-OH, CO-N-Me-Trp-OH, CO-Pro-OH, CO-N-Me-Gly-OH, CO-N-Me-Ser-OH, CO-N-Me-Thr-OH, CO-N-Me-Cys-OH, CO-N-Me-Tyr-OH, CO-N-Me-Asn-OH, CO-N-Me-Gln-OH, CO-N-Me-Asp-OH, CO-N-Me-Glu-OH, CO-N-Me-Lys-OH, CO-N-Me-Arg-OH, CO-N-Me-His-OH, CO-N-Me-Gly-Gly-OH, CO-N-Me-Gly-Gly-Gly-OH, CO-N-Me-Gly-Phe-OH, CO-N-Me-Gly-Glu-OH, CO-N-Me-Gly-Glu-Glu-OH, CO-N-Me-Glu-Glu-OH, CO-N-Me-Gly-Lys-OH, CO-N-Me-Gly-Lys-Lys-OH, CO-Pro-Glu-OH, CO-Pro-Glu-Glu-OH, CO-Pro-Gly-OH, CO-Pro-Gly-Lys-OH, and CO-Pro-Lys-Lys-OH;

### X is O or S; and

Z is substituted phenyl, or substituted 5- or 6-membered heterocyclic ring containing 1 or 2 heteroatoms chosen from N, O, and S, wherein said phenyl substituents are selected from the group consisting of H, lower alkyl, I, Br, Cl, F, CF₃, CH₂CF₃, CH₂Ph, CHPh₂, CH═CH₂, E-CH═CHCH₃, Z—CH═CHCH₃, C≡CH, C≡N, OCH₃, OCF₃, OPh, and NO₂ and wherein said heterocyclic ring substituents are selected from the group consisting of H, lower alkyl, I, Br, Cl, F, CF₃, CH₂CF₃, CH₂Ph, CH═CH₂, C≡CH, C≡N, OCH₃, OCF₃, Ph, OPh, and NO₂.

The term “pharmaceutically acceptable salts” refers to salts prepared from pharmaceutically acceptable non-toxic bases or acids including inorganic or organic bases and inorganic or organic acids. When the compound of the present invention is acidic, salts may be prepared from pharmaceutically acceptable non-toxic bases, including inorganic and organic bases. Salts derived from inorganic bases include aluminum, ammonium, calcium, copper, ferric, ferrous, lithium, magnesium, potassium, sodium, and zinc salts, and the like. Salts in the solid form may exist in more than one crystal structure, and may also be in the form of hydrates. Salts derived from pharmaceutically acceptable organic non-toxic bases include, but are not limited to, salts of primary amines, secondary amines, tertiary amines, substituted amines including naturally occurring substituted amines, cyclic amines, arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid(+), camphor-10-sulfonic acid (+), capric acid (decanoic acid), orotic acid, caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, isethionic acid, lactic acid (DL), lactobionic acid, lauric acid, maleic acid, malic acid (L), malonic acid, mandelic acid (DL), methanesulfonic acid, mucic acid, naphthalene-1,5-disulfonic acid, naphthalene-2-sulfonic acid, nicotinic acid, nitric acid, oleic acid, oxalic acid, palmitic acid, pamoic acid, pantothenic acid, phosphoric acid, propionic acid, pyroglutamic acid (L), salicylic acid, sebacic acid, stearic acid, succinic acid, sulfuric acid, tartaric acid (L), thiocyanic acid, p-toluenesulfonic acid, and undecylenic acid. It will be understood that, as used herein, compounds of Formula I are meant to also include the pharmaceutically acceptable salts.

The term “fused phenyl” refers to an compound in which two of the carbon atoms of a benzene ring are shared in a larger structure. Non-limiting examples include 1-naphthol, in which a benzene ring can be visualized as fused at carbons 2 and 3 of phenol, and 1,2,3,4-tetrahydronaphthaline, which can be visualized as benzene fused to cyclohexene.

The term “radical” refers to a chemical array of atoms which is bonded to another atom in a compound of the invention. The radical is a domain of a molecule described herein, and is not intended to be understood as a separate chemical entity, but rather a substituent or substituent array of atoms which is a part of a molecule of the invention. A radical as used herein is not intended to be understood to have ionic charge or singlet or triplet character, but rather covalently bonded to another domain of the compound of the invention. Stylistic depictions of radicals herein are intended solely to illustrate certain embodiments of the invention.

The term “lower alkyl” means straight-chain or branched hydrocarbon radicals containing 6 or fewer carbons. Examples include, but are not limited to, methyl, ethyl, propyl, butyl, isopropyl, and tert-butyl, signified by CH₃, CH₂CH₃, CH₂CH₂CH₃, (CH₂)₃CH₃, CH(CH₃)₂, and C(CH₃)₃, respectively. Further examples include cyclopropyl, cyclobutyl, cyclopentyl, and cyclohexyl.

Dialkylaminocarbonyl radicals, or secondary amine carbamoyl radicals, are those in which a dialkylamino substituent is joined to another atom by a carbonyl group, designated herein as CO. One example not intended to be limiting is diethylaminocarbonyl, in which N(CH₂CH₂)₂ is joined through a CO bond to an atom of another molecule, for example to the oxygen of phenol, to form a carbamate derivative. The product, C₆H₅OCONH(CH₂CH₃)₂, contains the CON(CH₂CH₂)₂ radical. A second non-limiting example is the 1-pyrrolidinecarbonyl radical, stylistically represented by A, when bonded to the oxygen of phenol, gives the carbamate product, which contains the 1-pyrrolidinecarbonyl radical.

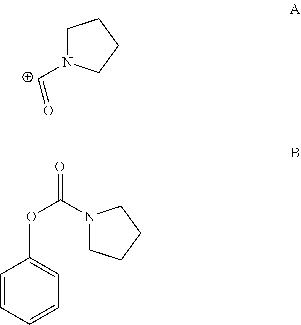

The secondary amine from which the dialkylaminocarbonyl radical is derived might be an N-alkyl amino acid. The terminology used herein to describe such radicals utilizes the well-known abbreviations for amino acids, coupled with widely used descriptors indicating N(α)-alkyl amino acids. The three letter codes for the naturally occurring amino acids (Eur. J. Biochem. 138:9-37 (1984)) are used herein. The secondary amine derived from naturally occurring amino acids, which gives rise to the said dialkylaminocarbonyl radicals, is described herein as an abbreviation encompassing the accepted three-letter amino acid abbreviations with the prefix N-Me. Thus, the secondary amine from which the dialkylaminocarbonyl radical is derived is referred to herein as N-Me-XXa-OH where Me denotes methyl and Xaa denotes generically any three-letter abbreviation for a naturally-occurring amino acid. The three-letter codes for the amino acids are widely know and can be found in Lehninger, Biochemistry, (Worth Publishers, New York, N.Y., 1978). The corresponding dialkylaminocarbonyl radical is referred to herein as CO-N-Me-Xaa-OH. As examples not intended to be limiting, the CO-N-Me-Ala-OH radical (wherein Xaa=Ala) has the stylized structure

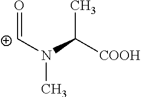

and the CO-N-Me-Leu-OH radical (wherein Xaa=Leu) has the stylized structure

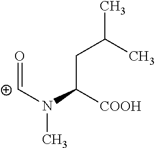

In likewise fashion, the secondary amine from which the dialkylaminocarbonyl radical is derived might be a di- or tri-peptide or di- or -tri-psuedopeptide, optionally with an N-alkylated N-terminus. For a dipeptide radical, the corresponding dialkylaminocarbonyl radical is referred to herein as CO-N-Me-Xaa-Xaa-OH. Thus, the CO-N-Me-Gly-Gly-OH radical can be represented as

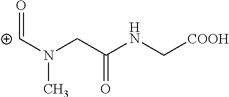

The stylized depictions of dialkylaminocarbonyl radicals herein are not meant to reflect actual chemical species, but are rather offered as a means to better understand the invention and the nomenclature used herein to describe the various embodiments. It will be understood that compounds of the invention containing amino acids refers to the natural (L) form, the (D) form, and the racemic (D,L) form.

In some embodiments, compounds of Formula I comprise

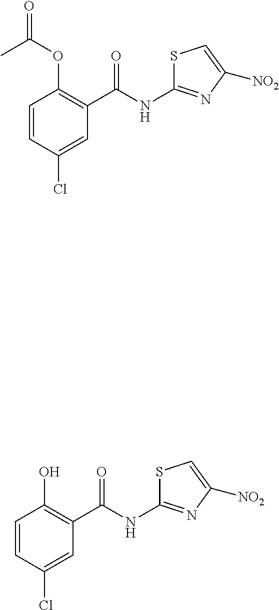 [SMILES: CC(=O)Oc1ccc(Cl)cc1C(=O)Nc1nc([N+](=O)[O-])cs1 ; O=C(Nc1nc([N+](=O)[O-])cs1)c1cc(Cl)ccc1O]

In some embodiments of the invention, compounds of Formula 1 comprise

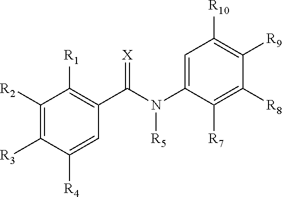

wherein

### R1=OH;

### R2 and R3=H;

R4 is selected from the set consisting of H, Cl, Br, F, CH₃, OCH₃, CF₃, OCF₃, and phenyl;

### R5=H; and

R7, R8, R9, and R10 are each independently chosen from the group consisting of Cl, Br, F, CH₃, CH₂CH₃, CH₂Ph, CH═CH₂, C≡CH, C≡N, OCH₃, OCF₃, Ph, OPh, and NO₂.

In some embodiments of the invention, compounds of Formula 1 comprise

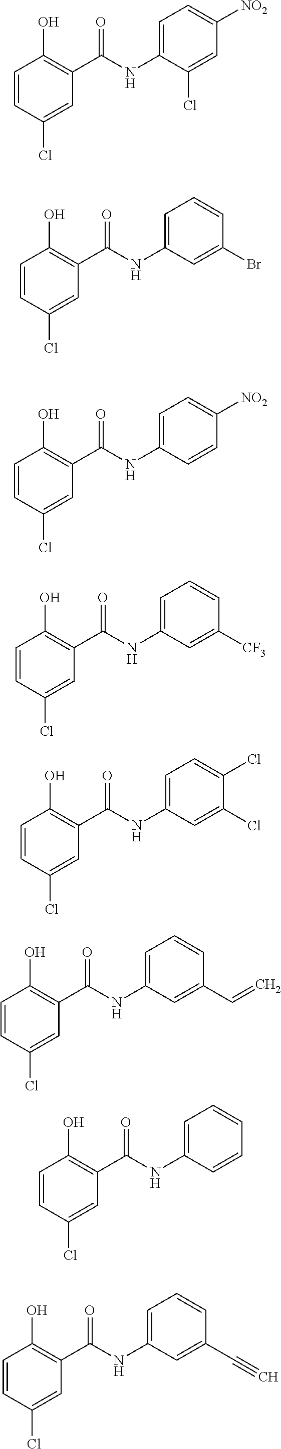

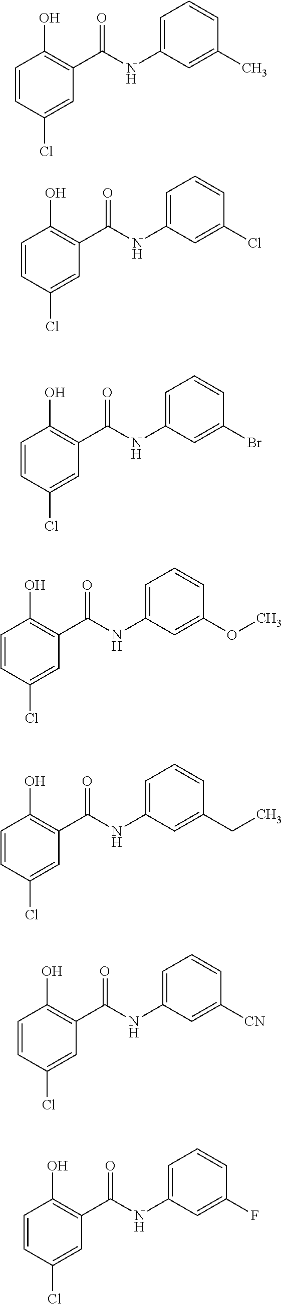

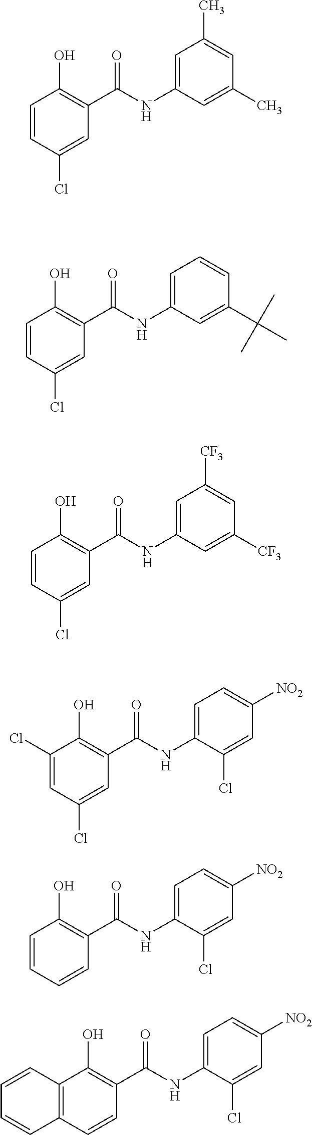 [SMILES: Cc1cc(C)cc(NC(=O)c2cc(Cl)ccc2O)c1 ; CC(C)(C)c1cccc(NC(=O)c2cc(Cl)ccc2O)c1 ; O=C(Nc1cc(C(F)(F)F)cc(C(F)(F)F)c1)c1cc(Cl)ccc1O ; O=C(Nc1ccc([N+](=O)[O-])cc1Cl)c1cc(Cl)cc(Cl)c1O ; O=C(Nc1ccc([N+](=O)[O-])cc1Cl)c1ccccc1O ; O=C(Nc1ccc([N+](=O)[O-])cc1Cl)c1ccc2ccccc2c1O]

Derivatives of active elements of Formula 1 which are designed to degrade in a controlled fashion under conditions of use of compounds of the invention, ultimately providing an active agent of Formula 1. These derivatives of the active compounds of Formula 1, herein termed “prodrugs”, are useful in that their chemical structure, although demonstrating little or no binding affinity, imparts properties of particular importance in the treatment of diseases or disorders mediated fully or partially by Group 1 mGluRs as disclosed herein. Such properties include, but are not limited to, enhancement of solubility in aqueous systems, improvement of pharmacokinetic parameters, improvement of purification procedures, enhancement of membrane permeability, and the provision of controlled release of the active principle. The degradation of the inert derivative to the active compound of Formula 1 may occur by simple chemical hydrolysis. Alternatively, said derivative may be a substrate for an enzyme which provides the active compound. Derivatives are chosen so that the chemical bond is cleavable under physiological conditions, whether chemical or enzymatic. In the case of the present invention, active compounds of Formula 1 possess a free phenolic —OH, and prodrug derivatives are esters or carbamates. It is well known in the art that certain inactive prodrug esters and carbamates of hydroxyl-containing drugs yield the drug after administration to a subject. For example, Dipivefrin, an inactive prodrug containing two tert-butyl esters, is cleaved to the drug adrenaline after administration to a subject, and valacyclovir, a valine ester, is cleaved to acyclovir after administration. For another example, the inactive prodrug bis(dimethylcarbamate) bambuterol is converted to the [beta]₂-sympathomimetic agent terbutaline used to achieve bronchodilation in the management of asthma. Terbutaline is formed from bambuterol by hydrolysis predominantly catalyzed by plasma cholinesterase (pChE, EC 3.1.1.8) (Nyberg, L. et al, Br. J. Clin. Pharmacol. 1998; 45(5); 471-8.

In some embodiments, prodrug compounds of Formula 1 comprise

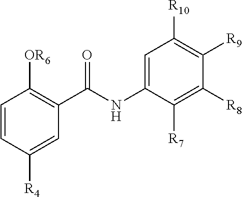

wherein
R4 is selected from the set consisting of H, Cl, Br, F, CH₃, OCH₃, CF₃, OCF₃, and phenyl;
R₆ is selected from the group consisting of COCH₃, COCH₂CH₃, COCH(CH₃)₂, COC(CH₃)₃, COPh, COCH₂Ph, COC₆H₄NO₂(p), COC₆H₄OH(p), COC₆H₄NH₂(p), CON(CH₃)₂, CON(CH₂CH₃)₂, CON(CH₃)(CH₂CH₃), CON(CH₂Ph)₂, CON(CH₃)(CH₂Ph), 1-pyrrolidinecarbonyl, 2-carboxy-1-pyrrolidinecarbonyl, (2S)-2-carboxy-1-pyrrolidinecarbonyl, (2R)-2-carboxy-1-pyrrolidinecarbonyl, 1-morpholinecarbonyl, 4-methyl-1-piperazinecarbonyl, sarcosine-N-carbonyl, CO-N-Me-Ala-OH, CO-N-Me-Val-OH, CO-N-Me-Leu-OH, CO-N-Me-Ile-OH, CO-N-Me-Val-OH, CO-N-Me-Met-OH, CO-N-Me-Phe-OH, CO-N-Me-Trp-OH, CO-Pro-OH, CO-N-Me-Gly-OH, CO-N-Me-Ser-OH, CO-N-Me-Thr-OH, CO-N-Me-Cys-OH, CO-N-Me-Tyr-OH, CO-N-Me-Asn-OH, CO-N-Me-Gln-OH, CO-N-Me-Asp-OH, CO-N-Me-Glu-OH, CO-N-Me-Lys-OH, CO-N-Me-Arg-OH, CO-N-Me-His-OH, CO-N-Me-Gly-Gly-OH, CO-N-Me-Gly-Gly-Gly-OH, CO-N-Me-Gly-Phe-OH, CO-N-Me-Gly-Glu-OH, CO-N-Me-Gly-Glu-Glu-OH, CO-N-Me-Glu-Glu-OH, CO-N-Me-Gly-Lys-OH, CO-N-Me-Gly-Lys-Lys-OH, CO-Pro-Glu-OH, CO-Pro-Glu-Glu-OH, CO-Pro-Gly-OH, CO-Pro-Lys-OH, CO-Pro-Lys-Lys-OH, CO-Pro-Gly-Lys-OH, and CO-Pro-Lys-Lys-OH; and
R7, R8, R9, and R10 are each independently chosen from the group consisting of Cl, Br, F, CH₃, CH₂CH₃, CH₂Ph, CH═CH₂, C≡CH, C≡N, OCH₃, OCF₃, Ph, OPh, and NO₂.

It will be appreciated that other amino acids not specifically identified herein might be utilized in prodrug compounds of Formula 1. These may be selected from the family of naturally occurring L-amino acids, that is, alanine, valine, leucine, isoleucine, proline, phenylalanine, tryptophan, methionine, glycine, serine, threonine, cysteine, cystine, tyrosine, asparagine, glutamine, aspartic acid, glutamic acid, lysine, arginine, and histidine. It will also be appreciated that, in addition to the L-amino acids herein described, unnatural D-amino acids may be employed in prodrug compounds of Formula 1. It will be further appreciated that, in addition to the specific prodrugs of Formula 1 disclosed herein, other peptide prodrugs are contemplated by the present invention. These include, but are not limited to, di-, tri-, tetra-, penta-, hexa-, hepta, octa-, nona-, and deca-peptides comprised of any of the natural L-amino acids or the unnatural D-amino acids or their N-methyl derivatives.

In some embodiments, prodrug compounds of Formula 1 comprise

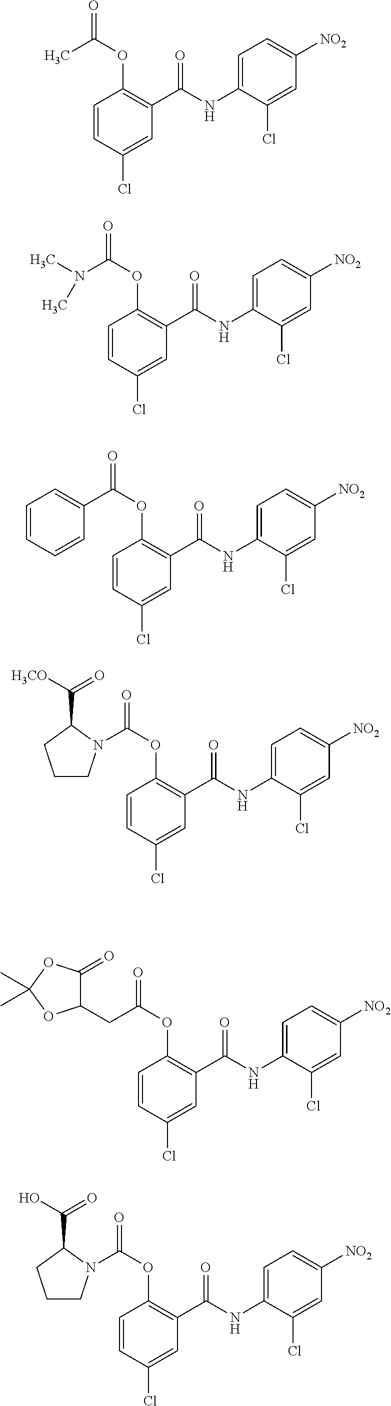

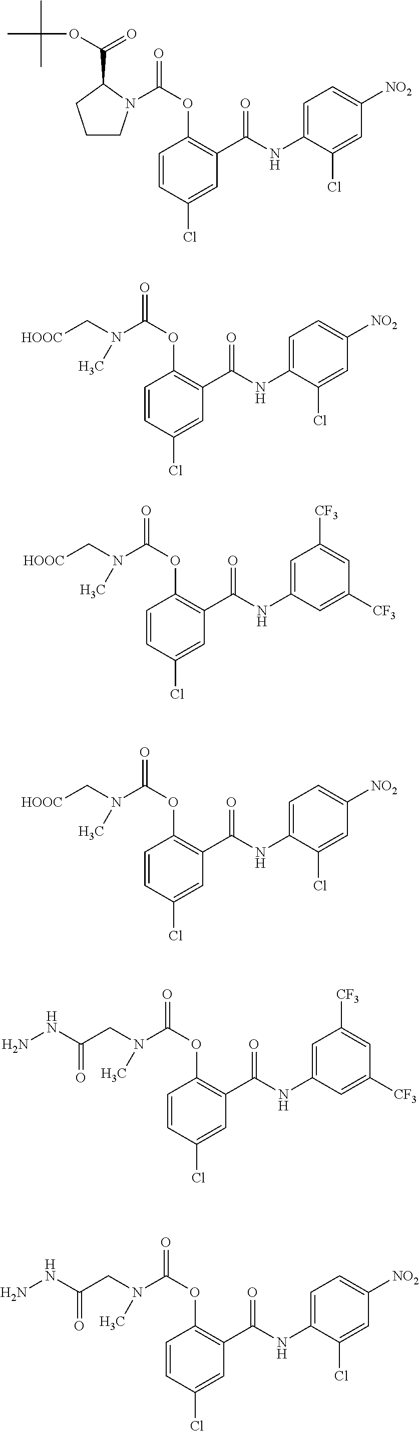 [SMILES: CC(C)(C)OC(=O)[C@@H]1CCCN1C(=O)Oc1ccc(Cl)cc1C(=O)Nc1ccc([N+](=O)[O-])cc1Cl ; CN(CC(=O)O)C(=O)Oc1ccc(Cl)cc1C(=O)Nc1ccc([N+](=O)[O-])cc1Cl ; CN(CC(=O)O)C(=O)Oc1ccc(Cl)cc1C(=O)Nc1cc(C(F)(F)F)cc(C(F)(F)F)c1 ; CN(CC(=O)O)C(=O)Oc1ccc(Cl)cc1C(=O)Nc1ccc([N+](=O)[O-])cc1Cl ; CN(CC(=O)NN)C(=O)Oc1ccc(Cl)cc1C(=O)Nc1cc(C(F)(F)F)cc(C(F)(F)F)c1 ; CN(CC(=O)NN)C(=O)Oc1ccc(Cl)cc1C(=O)Nc1ccc([N+](=O)[O-])cc1Cl]

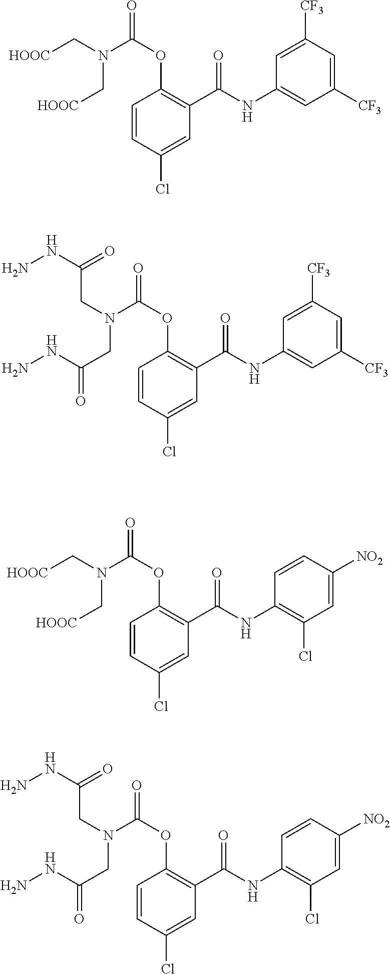 [SMILES: O=C(O)CN(CC(=O)O)C(=O)Oc1ccc(Cl)cc1C(=O)Nc1cc(C(F)(F)F)cc(C(F)(F)F)c1 ; NNC(=O)CN(CC(=O)NN)C(=O)Oc1ccc(Cl)cc1C(=O)Nc1cc(C(F)(F)F)cc(C(F)(F)F)c1 ; O=C(O)CN(CC(=O)O)C(=O)Oc1ccc(Cl)cc1C(=O)Nc1ccc([N+](=O)[O-])cc1Cl ; NNC(=O)CN(CC(=O)NN)C(=O)Oc1ccc(Cl)cc1C(=O)Nc1ccc([N+](=O)[O-])cc1Cl]

Prodrug derivative compounds of Formula 1 can be transformed into pharmaceutically acceptable salts by means well known to those with skill in the art. Such salts are intended to provide enhanced properties such as increased water solubility. Such salts are contemplated by the invention and provided herein. Such salts may be prepared by methods well-known to those with skill in the art. For example, an acidic prodrug derivative compound of Formula 1 may be dissolved in a solvent such as, inter alia, methanol, ethanol, or tetrahydrofuran and a molar equivalent amount of meglumine added to form the addition salt. Removal of the solvent or precipitation of the salt by addition of a cosolvent such as, inter alia, ether, petroleum ether, hexane, heptane, or toluene provides the purified salt. Alternatively, such salts may be prepared by methods disclosed in U.S. Pat. No. 5,028,625 to Motola et al. Alternatively, such salts may be prepared by methods disclosed in WO/2007/063335 to Klaveness.

In some embodiments of the invention, pharmaceutically acceptable salts of prodrug derivative compounds of Formula 1 comprise

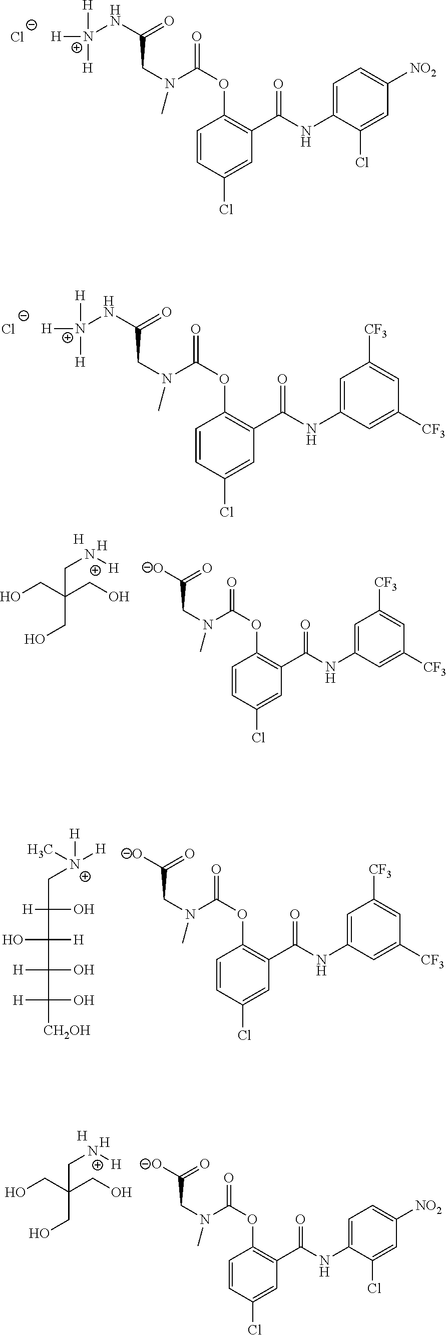

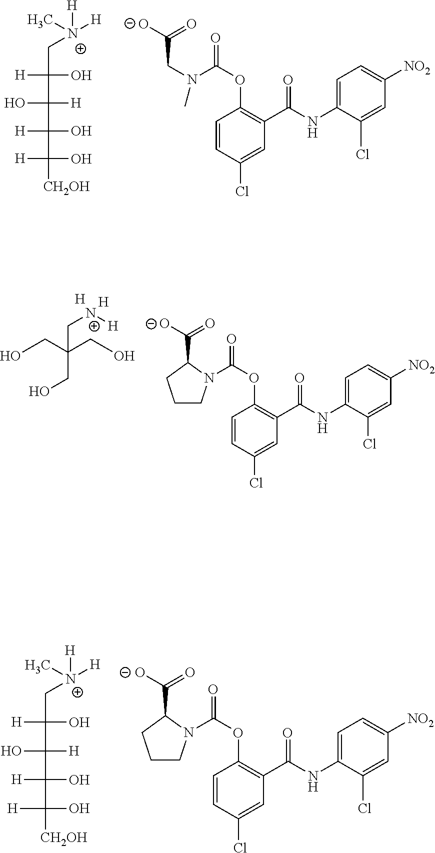

Compounds of Formula I are useful in treating apicomplexan infections. Apicomplexan infections can afflict animals including, but not limited to, humans, domestic pets, and livestock. The invention is directed to contacting an animal in need of treatment with an effective amount of a composition comprising one or more of the compounds of the invention. As used herein, the terms “treatment” and “treating” refer to any process wherein there may be a slowing, interrupting, arresting, controlling, ameliorating, lessening, regulating, or stopping of the progression of the disorders caused wholly or in part by infection with an apicomplexan including, but not limited to those described herein, but does not necessarily indicate a total elimination of all symptoms of the disorders. “Treatment” and “treating” may also refer to prophylactic therapy of the disorders caused wholly or in part by infection with an apicomplexan including, but not limited to those described herein.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing one or more compounds of Formula I and a pharmaceutically acceptable carrier. By “pharmaceutically acceptable” it is meant the carrier, diluent or excipient must be devoid of intrinsic biological activity, and be compatible with the other ingredients of the formulation and not deleterious to the recipient thereof.

The terms “administration of” and or “administering” a compound should be understood to mean providing a compound of Formula I or a prodrug of a compound of Formula I or a composition containing a compound or prodrug of a compound of Formula 1 to an individual in need of treatment by a route generally accepted by those with skill in the art. Routes of such administration include, but are not limited to, oral, buccal, sublingual, inhalation, topical, ocular, transcutaneous, intravenous, subcutaneous, intraperitoneal, transdermal, intracerebroventricular, intrathecal, intracerebral implant, and depot implant.

Compounds of Formula I may be prepared by methods known to those with skill in the art. Commercially available salicylic acids 1 were coupled with commercially available anilines 2 in hot xylenes in the presence of PCl₃ to furnish salicylanilides 3 (U.S. Pat. No. 307,927) (Scheme 1 and Table 1).

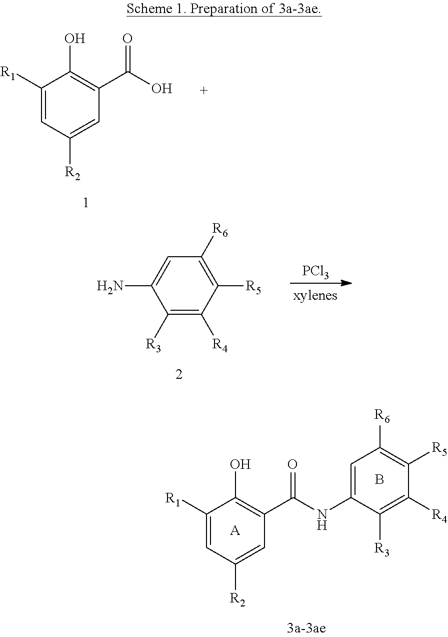

Reduction of niclosamide 4 with Zn dust in methanol and acetic acid followed by salt formation gave amino salicylanilide hydrochloride 5 (Scheme 2).

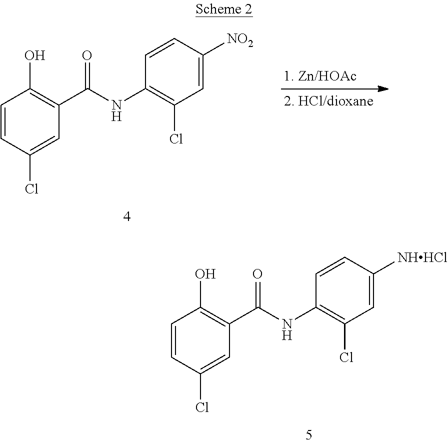

Simple ester or carbamate derivatives of 4 were obtained through treatment of 4 with various carbonyl chlorides 6 to provide acylated derivatives 7 (Scheme 3 and Table 2).

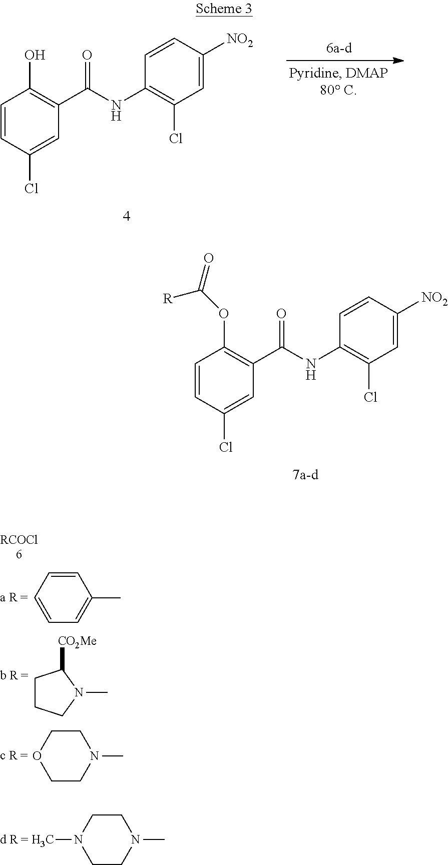

The fluorine-containing salicylanilide methyl ethers 10 were synthesized by HATU-mediated condensation of 5-fluoro-2-methoxybenzoic acid 8 with nitroanilines 9 (Scheme 4).

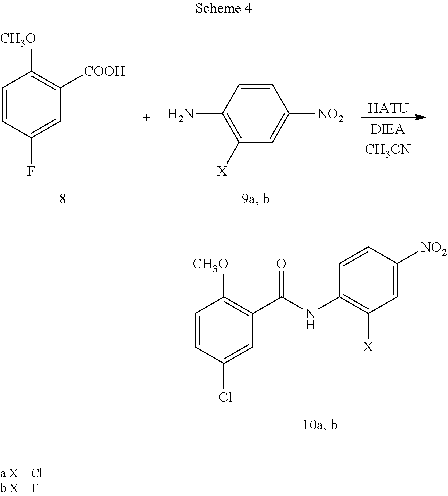

Sarcosine tert-butyl ester hydrochloride 11 was transformed into the free base and treated with phosgene in toluene to provide tert-butyl 2-((chlorocarbonyl)(methyl)amino)acetate 12. We found that triphosgene and a solution of phosgene in toluene are essentially equivalent for this transformation. 12 reacted with 4 smoothly in warm pyridine under DMAP catalysis to furnish carbamate ester 13a. Sequential removal of the ester function by treatment with trifluoroacetic acid, condensation of the resulting carboxylic acid with tert-butyl carbazate using EDCI, and treatment of the resulting protected acid hydrazide with HCl in dioxane furnished the sarcosine hydrazide hydrochloride 14a. In a similar fashion, salicylanilide 3j was converted to the corresponding sarcosine hydrazide hydrochloride 14b (Scheme 5).

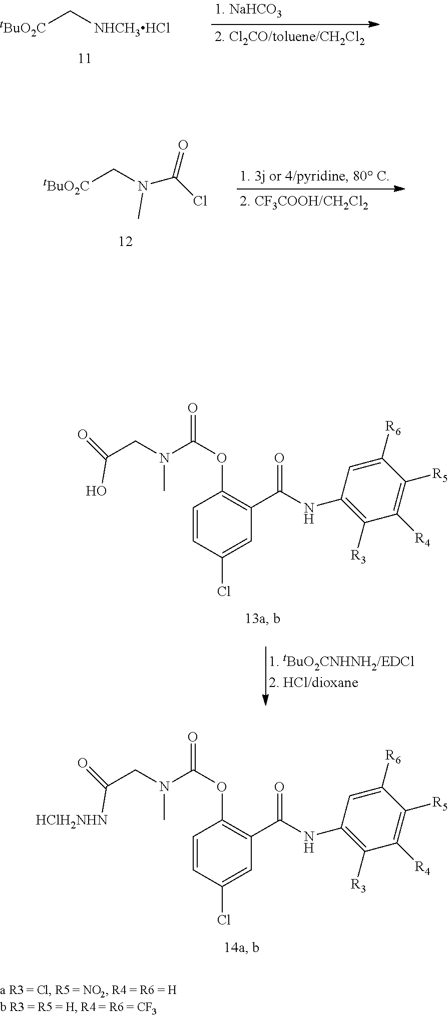

Determination of the anti-apicomplexan activity of Compounds of Formula I may be determined by one skilled in the art. Parasite proliferation was monitored by using stably transfected type I RH-YFP parasites, which constitutively express Yellow Florescent Protein. Proliferation also was tested using a [³H]-Uracil incorporation assay, as Uracil is incorporated into nucleic acids of T. gondii tachyzoites, but not mammalian cells, as they divide. Complimentary challenge assays ensured that the observed fluorescence data was due to parasite inhibition and not to quenched fluorescence. Pyrimethamine and sulfadiazine were used as positive controls, and DMSO at a concentration of 0.1% was used as the negative control. Because T. gondii is an obligate parasite, compounds that are toxic to host cells will appear to inhibit parasite growth. Therefore, all test compounds were simultaneously evaluated for efficacy and toxicity against human cells.

Selected compounds exhibiting the ability to inhibit tachyzoite growth were evaluated to determine whether their activity was due to a static or cidal effect.

Compound activity against P. falciparum, the causative agent of malaria, was assessed using the Malaria SYBR Green 1-Based Fluorescence (MSF) Assay. This microtiter plate drug sensitivity assay uses the presence of malarial DNA as a measure of parasitic proliferation. The assay is a microtiter plate drug sensitivity assay that uses the presence of malarial DNA as a measure of parasitic proliferation in the presence of antimalarial drugs or experimental compounds based on modifications of previously described methods (Plouffe et al., Proc Natl Acad Sci USA. 2008, 105, 9059-9064), (Johnson et al., Antimicrob. Agents Chemother. 2005, 49, 3463-3467). As the intercalation of SYBR Green I dye and its resulting fluorescence is relative to parasite growth, a test compound that inhibits the growth of the parasite will result in a lower fluorescence.

Selected compounds were evaluated for in vivo efficacy against T. gondii and P. falciparum. To asses toxic effects when administered orally, selected compounds were administered orally to mice daily for nine days at a dose of 100 mg/kg. At the end of the ten days, the animals were evaluated for toxic effects.

Compounds 14a and 14b were tested for efficacy against T. gondii in the oocyst stage following per oral challenge in mice. Mice were infected by oral gavage with ME49 or TgGoatUS4 oocysts. Mice were treated with either 100 mg/kg or 25 mg/kg) of test substance.

Selected compounds were examined for activity against two strains of P. falciparum: D6 (CDC/Sierra Leone), a drug-sensitive and readily killed by chloroquine, and TM90-C235, a multi-drug resistant strain resistant to chloroquine.

Salicylanilides 3a-3ae, 4, 5, and derivatives 7a-7d, 14 a, and 14b were tested for in vitro efficacy against T. gondii tachyzoites. It was decided that only those compounds possessing MIC₅₀≦1 μM would be considered active against T. gondii. The efficacy and corresponding cellular toxicity data appear in Table 1 (salicylanilides 3a-ae, 4, and 5), Table 2 (acylated salicylanilides 7), and Table 3 (ionized derivatives 13b and 14b). Of the 39 compounds assayed, 16 (41%) had MIC₅₀≦1 μM, 12 (31%) had MIC₅₀≦500 nM, 6 (15%) had MIC₅₀≦250 nM, and 4 (10%) had MIC₅₀≦125 nM. This limited data set suggests that several closely related salicylanilide derivatives show promising activity but the core salicylanilide chemotype does not represent a non-specific inhibition of the parasite.

The discovery of the activity of niclosamide 4 led to a limited medicinal chemistry effort to probe the effects of the variation of ring substituents and the derivitization of the phenolic oxygen in various ways. Tables 1 and 3 outline the various structures and activities of the salicylanilides evaluated. Initially the phenolic functionality was maintained, and two alterations to the A (salicyl) ring were made while retaining the 2′-chloro-4′-nitro B (anilide) ring. Replacement of the 5-chloro of 4 with methyl (3m) decreased potency, while replacement with H (3k) eliminated activity altogether. Compounds with a 4-fluoro substituent (3ac, 3ad, and 3 ae) or 3,5-diiodo substitution (3aa) demonstrated no activity. The decision was made to proceed with the study of 4-chloro A-ring analogs with a variety of B ring substituents.

TABLE 1
Inhibitory Activity of Salicylanilides against Toxoplasmosis gondii. Compounds do not
exhibit toxic effects to host cells (IC₅₀ > 1 μM).

| Structure | Compound | MW | MIC₅₀ | | MIC₉₀ | |
|---|---|---|---|---|---|---|
| 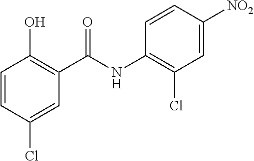 [SMILES: O=C(Nc1ccc([N+](=O)[O-])cc1Cl)c1cc(Cl)ccc1O] | 4 | 327.12 | 250-200 | nM | 250-200 | nM |
| 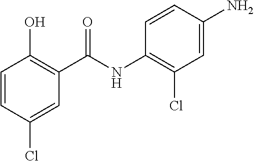 [SMILES: Nc1ccc(NC(=O)c2cc(Cl)ccc2O)c(Cl)c1] | 5 | 297.14 | >1 | μM | >1 | μM |
| 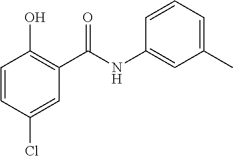 [SMILES: Cc1cccc(NC(=O)c2cc(Cl)ccc2O)c1] | 3a | 261.70 | >1 | μM | >1 | μM |
| 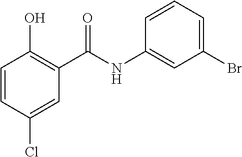 [SMILES: O=C(Nc1cccc(Br)c1)c1cc(Cl)ccc1O] | 3b | 326.57 | 750-500 | nM | 1 μM-750 | nM |
| 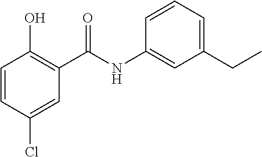 [SMILES: CCc1cccc(NC(=O)c2cc(Cl)ccc2O)c1] | 3c | 275.73 | 500-250 | nM | 750-500 | nM |
| 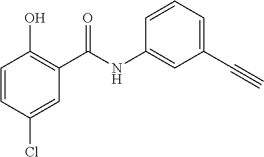 [SMILES: C#Cc1cccc(NC(=O)c2cc(Cl)ccc2O)c1] | 3d | 271.70 | >1 | μM | >1 | μM |
| 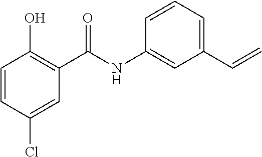 [SMILES: C=Cc1cccc(NC(=O)c2cc(Cl)ccc2O)c1] | 3e | 273.71 | 500-250 | nM | 1 μM-750 | nM |
| 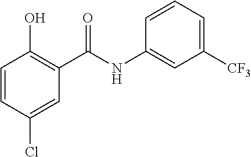 [SMILES: O=C(Nc1cccc(C(F)(F)F)c1)c1cc(Cl)ccc1O] | 3f | 315.67 | 500-250 | nM | 500-250 | nM |
| 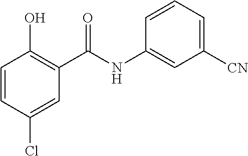 [SMILES: N#Cc1cccc(NC(=O)c2cc(Cl)ccc2O)c1] | 3g | 272.69 | >1 | μM | >1 | μM |
| 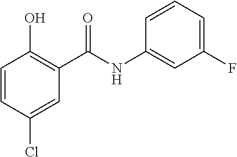 [SMILES: O=C(Nc1cccc(F)c1)c1cc(Cl)ccc1O] | 3h | 265.67 | 570-500 | nM | 1 μM-750 | nM |
| 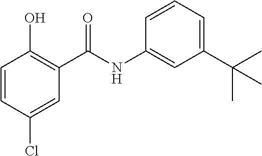 [SMILES: CC(C)(C)c1cccc(NC(=O)c2cc(Cl)ccc2O)c1] | 3i | 308.78 | 16-8 | nM | 31-16 | nM |
| 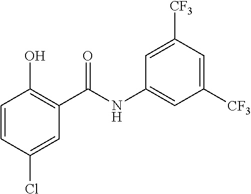 [SMILES: O=C(Nc1cc(C(F)(F)F)cc(C(F)(F)F)c1)c1cc(Cl)ccc1O] | 3j | 383.67 | 31-16 | nM | 250-125 | nM |
| 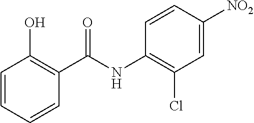 [SMILES: O=C(Nc1ccc([N+](=O)[O-])cc1Cl)c1ccccc1O] | 3k | 292.67 | >1 | μM | >1 | μM |
| 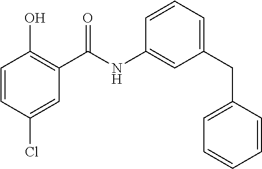 [SMILES: O=C(Nc1cccc(Cc2ccccc2)c1)c1cc(Cl)ccc1O] | 3l | 337.80 | 1 uM-750 | nM | 1 μM-750 | nM |
| 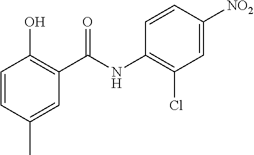 [SMILES: Cc1ccc(O)c(C(=O)Nc2ccc([N+](=O)[O-])cc2Cl)c1] | 3m | 306.70 | 500-250 | nM | 500-250 | nM |
| 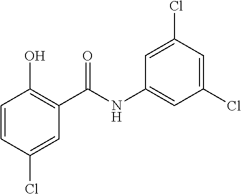 [SMILES: O=C(Nc1cc(Cl)cc(Cl)c1)c1cc(Cl)ccc1O] | 3n | 316.57 | 500-250 | nM | 500-250 | nM |
| 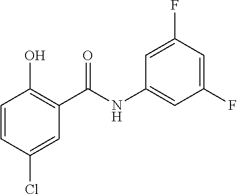 [SMILES: O=C(Nc1cc(F)cc(F)c1)c1cc(Cl)ccc1O] | 3o | 283.66 | >1 | μM | >1 | μM |
| 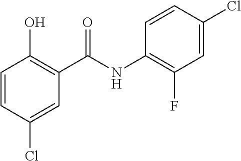 [SMILES: O=C(Nc1ccc(Cl)cc1F)c1cc(Cl)ccc1O] | 3p | 300.11 | 750-500 | nM | 750-500 | nM |
| 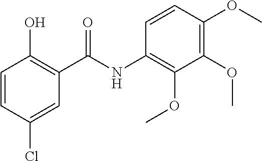 [SMILES: COc1ccc(NC(=O)c2cc(Cl)ccc2O)c(OC)c1OC] | 3q | 337.75 | >1 | μM | >1 | μM |
| 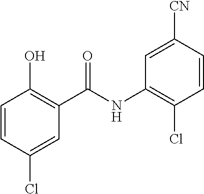 [SMILES: N#Cc1ccc(Cl)c(NC(=O)c2cc(Cl)ccc2O)c1] | 3r | 307.13 | 750-500 | nM | 1 μM-750 | nM |
| 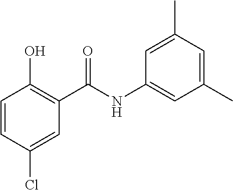 [SMILES: Cc1cc(C)cc(NC(=O)c2cc(Cl)ccc2O)c1] | 3s | 275.73 | >1 | μM | >1 | μM |
| 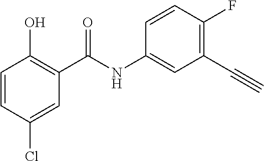 [SMILES: C#Cc1cc(NC(=O)c2cc(Cl)ccc2O)ccc1F] | 3t | 289.69 | >1 | μM | >1 | μM |
| 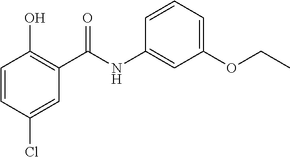 [SMILES: CCOc1cccc(NC(=O)c2cc(Cl)ccc2O)c1] | 3u | 291.73 | >1 | μM | >1 | μM |
| 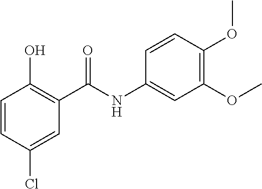 [SMILES: COc1ccc(NC(=O)c2cc(Cl)ccc2O)cc1OC] | 3v | 307.73 | >1 | μM | >1 | μM |
| 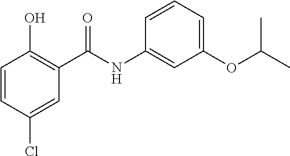 [SMILES: CC(C)Oc1cccc(NC(=O)c2cc(Cl)ccc2O)c1] | 3w | 305.76 | >1 | μM | >1 | μM |
| 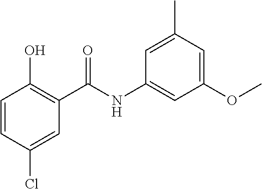 [SMILES: COc1cc(C)cc(NC(=O)c2cc(Cl)ccc2O)c1] | 3x | 291.73 | >1 | μM | >1 | μM |
| 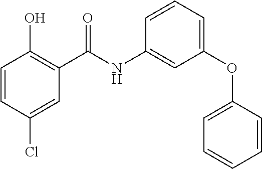 [SMILES: O=C(Nc1cccc(Oc2ccccc2)c1)c1cc(Cl)ccc1O] | 3y | 339.77 | >1 | μM | >1 | μM |
| 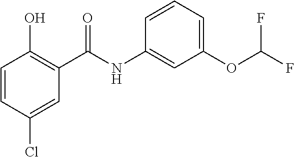 [SMILES: O=C(Nc1cccc(OC(F)F)c1)c1cc(Cl)ccc1O] | 3z | 313.68 | >1 | μM | >1 | μM |
| 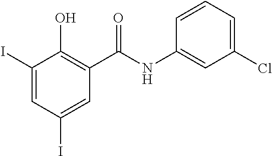 [SMILES: O=C(Nc1cccc(Cl)c1)c1cc(I)cc(I)c1O] | 3aa | 499.47 | >1 | μM | >1 | μM |
| 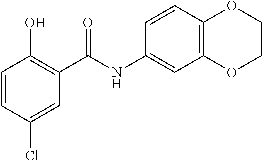 [SMILES: O=C(Nc1ccc2c(c1)OCCO2)c1cc(Cl)ccc1O] | 3ab | 305.71 | >1 | μM | >1 | μM |
| 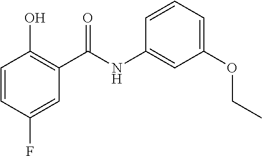 [SMILES: CCOc1cccc(NC(=O)c2cc(F)ccc2O)c1] | 3ac | 275.27 | >1 | μM | >1 | μM |
| 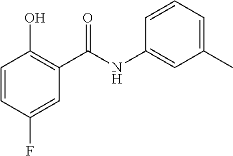 [SMILES: Cc1cccc(NC(=O)c2cc(F)ccc2O)c1] | 10a | 245.25 | >1 | μM | >1 | μM |
| 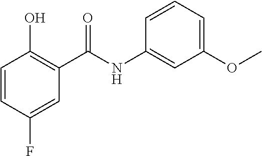 [SMILES: COc1cccc(NC(=O)c2cc(F)ccc2O)c1] | 10b | 261.25 | >1 | μM | >1 | μM |

Altering the electron-withdrawing character of the B ring substituents of 4 had a profound effect on activity. When a nitro group was replaced with an amino group at position 4′, (5) all activity was lost. Likewise, compounds possessing O-alkyl electron-donating substituents at 2′ or 4′ (3q, 3v, 3ab) were devoid of activity. It was surprising to note that replacement of the 2′ chloro with the more electronegative fluoro substituent, while simultaneously replacing the 4′ nitro with the less powerful electron withdrawing chloro group (3p), removed all activity.

A series of 3′monosubstituted compounds were examined. Various activities were observed, and it is clear that the nature of the substituent at this position has a profound effect. In this series, the activity range shows ^{t}Bu (3i)>>Et(3c)≈CH₂CH₂(3e)≈CF₃(3f)≈F(3h)>Br (3b)≈CH₂Ph (3l). All other 3′ substituents resulted in compounds with no activity. Clearly the introduction of 3′-alkoxy or aryloxy substitution resulted in no increase in activity and actually may even be detrimental. When compared to 3i, the best in the 3′-monosubstituted series, both electronegative (halo, CF₃) and modestly electron-donating (alkyl) substitutions provided moderate activity.

The activity of a few of the 3′-monosubstituted salicylanilides prompted the evaluation of four compounds with substituents at both the 3′ and 5′ positions. Compounds 3o (3′,5′-difluoro) and 3t (3′,5′-dimethyl) were inactive, and 3n (3′,5′-dichloro) showed slight activity. Interestingly, the 3′,5′-bis(trifluoromethyl) derivative 3j displayed promising activity.

A cursory study of the effect of capping the phenol of 4 via acylation was undertaken. The acylated derivatives 7 were prepared and tested. The structure and activity data are presented in Table 2. The carbamates 7b, 7c, and 7d, which impart altered polarity and hydrogen bonding capabilities compared to 4, were devoid of activity. We were surprised to learn that benzoate ester 7a showed an apparent increase in potency over 4. In other studies, we found that 7a and other carboxylic esters of 4 were hydrolytically labile under certain conditions (data not shown). Any differential activity of 7a over 4 therefore may be due to altered solubility and permeability parameters which 7a may possess, and that eventual liberation of active 4 may be responsible for enhancing the observed activity. The intrinsic activity of intact 7a cannot yet be ruled out, and this interesting phenomenon is currently under study. The carbamates 7b, 7c, and 7d are much more stable against hydrolysis (data not shown), and are not expected to yield free 4 during bioassay. The fact that these derivatives have no activity suggests that either the increased steric demand of the carbamate groups, or the capping of the phenolic oxygen, renders these compounds inactive.

TABLE 2
Inhibitory Activity of Acylated Salicylanilide Derivatives against T. gondii. Compounds do
not exhibit toxic effects to host cells (IC₅₀ > 1 μM).

| Structure | Compound | MW | MIC₅₀ | | MIC₉₀ | |
|---|---|---|---|---|---|---|
| 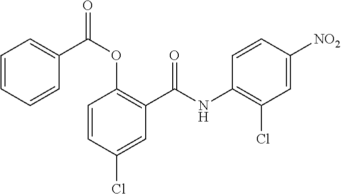 [SMILES: O=C(Oc1ccc(Cl)cc1C(=O)Nc1ccc([N+](=O)[O-])cc1Cl)c1ccccc1] | 7a | 432.23 | 125-61 | nM | 250-125 | nM |
| 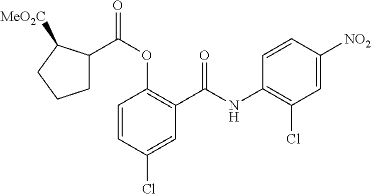 [SMILES: COC(=O)[C@@H]1CCCC1C(=O)Oc1ccc(Cl)cc1C(=O)Nc1ccc([N+](=O)[O-])cc1Cl] | 7b | 482.27 | >1 | μM | >1 | μM |
| 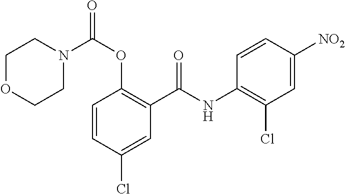 [SMILES: O=C(Nc1ccc([N+](=O)[O-])cc1Cl)c1cc(Cl)ccc1OC(=O)N1CCOCC1] | 7c | 261.7 | >1 | μM | >1 | μM |
| 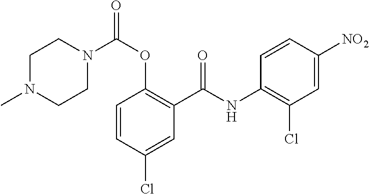 [SMILES: CN1CCN(C(=O)Oc2ccc(Cl)cc2C(=O)Nc2ccc([N+](=O)[O-])cc2Cl)CC1] | 7d | 326.57 | 750-500 | nM | 1 μM-750 | nM |

Two 5-fluorosalicylanilide methyl ethers (10a and 10b) were synthesized and tested, and proved to be inactive. This series of salicylanilide ethers was not further pursued.

The acid hydrazide salts 14a and 14b were designed to possess enhanced solubility and bioavailability relative to the parent structures. We were delighted to learn that 14a and 14b possess compelling in vitro (Table 3) and in vivo activity against a highly virulent challenge.

TABLE 3
Inhibitory Activity of Ionized Salicylanilide Derivatives against Toxoplasmosis gondii. Compounds
do not exhibit toxic effects to host cells (IC₅₀ > 1 μM)

| Structure | Compound | MW | MIC₅₀ | | MIC₉₀ |
|---|---|---|---|---|---|
| 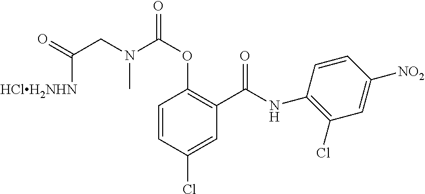 | 14a | 492.7 | 31-16 | nM | 250-125 nM |
| 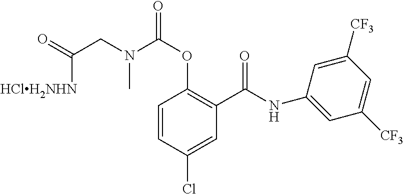 | 14b | 549.25 | 250-125 | nM | 250-125 nM |

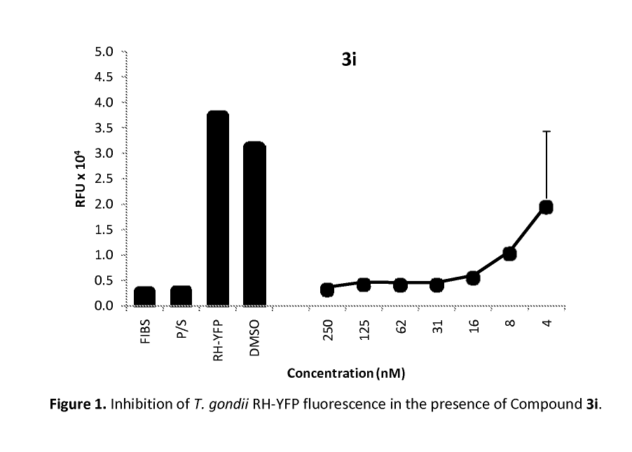

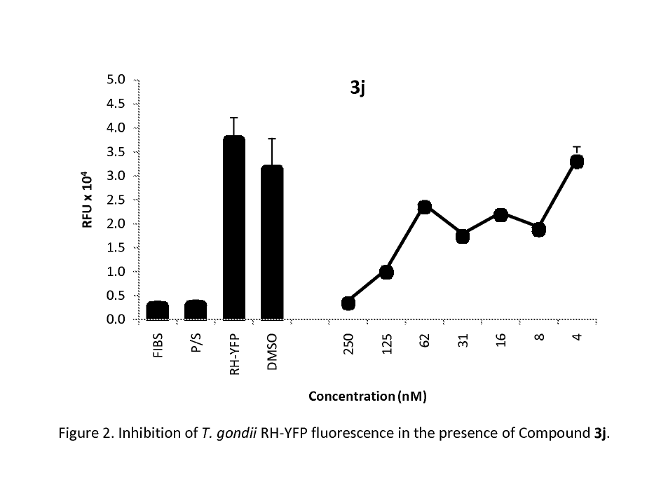

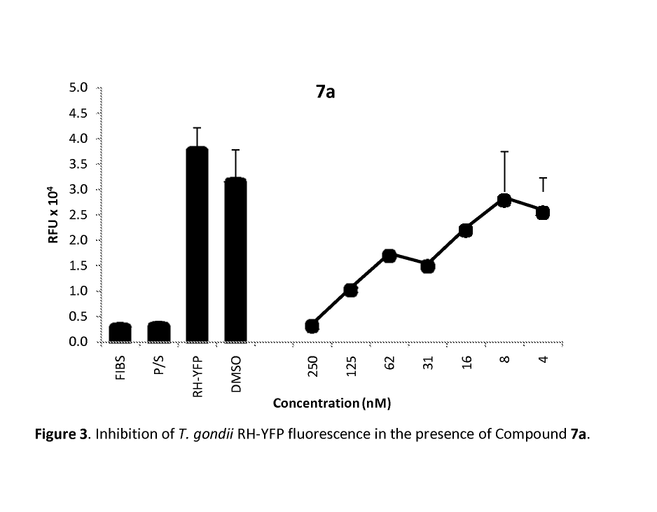

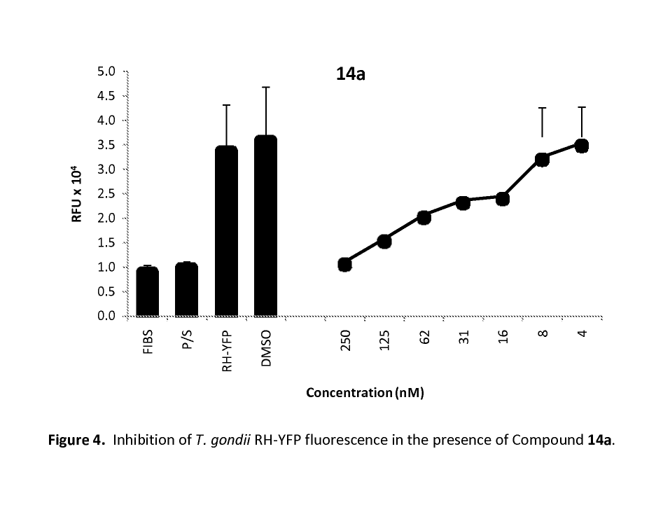

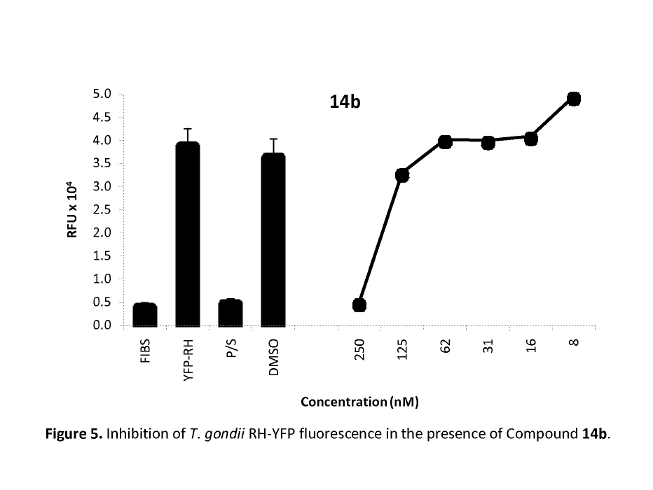

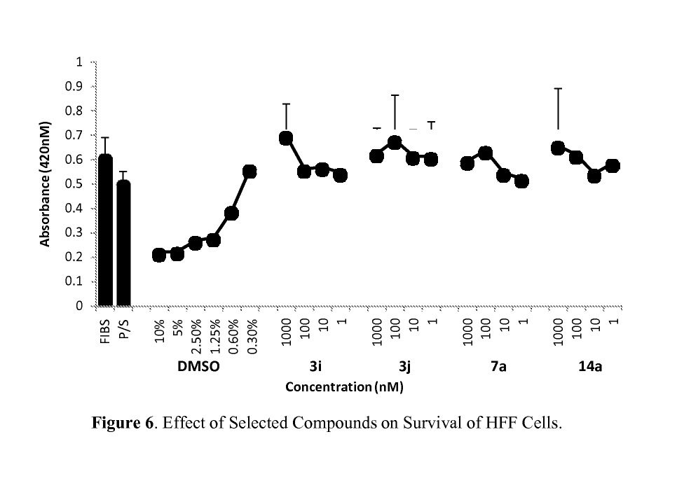

The screening efforts revealed that six of the compounds were the most effective inhibitors. Of these, 3i, 3j, 7a, 14a, and 14b were selected for further in vitro evaluation. Serial dilutions of these compounds to give additional test concentrations were made and tested to identify inhibitory IC₅₀ and IC₉₀values. The measured IC₅₀ and IC₉₀ ranges and the corresponding toxicity data appear in Table 4. The graphical presentation of parasite inhibition appears in FIGS. 1 through 5, while the graphical display of toxicity to HFF cells appears in FIG. 6. FIBS, host fibroblasts alone, not infected; P/S, infected control treated with pyrimethamine and sulfadiazine in combination; RH-YFP, untreated infected fibroblast control; 0.1% DMSO (vehicle) infected fibroblast control; [nM], concentration of the inhibitor dissolved in 0.1% DMSO.

TABLE 4
IC₅₀ and IC₉₀ Values of Selected Compounds

| Compound | IC₅₀, nM | IC₉₀, nM | Toxicity, nM |
|---|---|---|---|
| 3i | 160-08 | 31-16 | >1000 |
| 3j | 31-16 | 250-125 | >1000 |
| 7a | 125-61 | 250-125 | >1000 |
| 14a | 31-16 | 250-125 | >1000 |
| 14b | 250-125 | 250-125 | >1000 |

Since the ideal antiparasitic agent would have cidal activity, it is of interest whether potential antiparasitic drugs exhibit a static (inhibition of growth and/or replication) or cidal (lethal) effect. In order to determine whether leading compounds in this study inhibited parasite proliferation by either a cidal or static mechanism, four were selected (3i, 3j, 7a, and 14a) and applied at four to eight times MICs₅₀ to parasites. In this assay, RH-YFP tachyzoites were treated with each compound at 1 μM under various dosing conditions:

Condition A: Parasites were treated for four days, then compound was removed

Condition B: Parasites were treated for ten days, then compound was removed

Condition C: Compound was refreshed at four days then removed at ten days

Condition D: Compound was maintained for the duration of the experiment

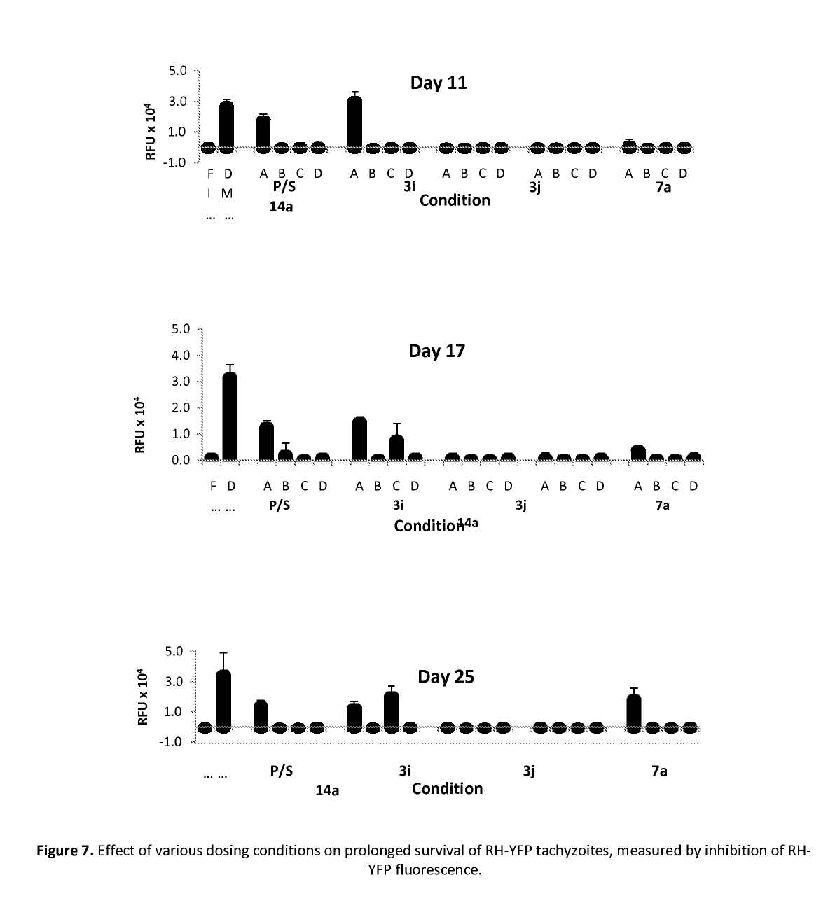

The four and ten day time points were taken to reveal the impact of extended exposure of the parasites to the test substance. Compounds were refreshed at four days to examine whether compound degradation could contribute to an observed static effect. Parasite growth was assessed at days 11, 17, and 25. The growth data, as a function of ³H-uracil uptake, is expressed in FIG. 7. 4 days, Condition A; 10 days, renewed at 4, Condition B; 10 days, Condition C; All time, Condition D. Ordinate: Counts per minute.

Treatment with 3i under Condition A reveals that the parasite burden is roughly equivalent to utreated controls at day 11. At day 17, Condition C dosing of 3i also shows renewed growth. Application of 3i under Condition D demonstrates inhibition. These data suggest that 3i is parasitostatic. Compounds 3j and 7a inhibited growth under all Conditions employed in this experiment. No parasite growth observed after the removal of these compounds, even at day 25, suggesting that their activity is parasitocidal. Compounds 3j and 7a demonstrated a cidal effect after four days of treatment, while 14a demonstrated a cidal effect after ten days of treatment, comparable to treatment with the combination of pyrimethamine and sulfadiazine.

The effect of selected compounds on other apicomplexan parasites was also determined.

Compounds 3i, 3j, 7a, and 14a were examined for activity against two strains of P. falciparum, the causative agent of malaria. One of these strains, D6 (CDC/Sierra Leone), is drug-sensitive and readily killed by chloroquine, while the second strain, TM90-C235, is multi-drug resistant and shows resistance to chloroquine. The activity of these compounds was assessed using the Malaria SYBR Green 1-Based Fluorescence (MSF) Assay. This microtiter plate drug sensitivity assay uses the presence of malarial DNA as a measure of parasitic proliferation. As shown in Table 5, all compounds demonstrated activity against both P. falciparum strains, with 7a the most effective (D6 chloroquine sensitive IC₅₀=295 ng/mL, 0.7 nM) and TM90-C235 chloroquine resistant IC₅₀=267 ng/mL, 0.6 nM). Compounds 7a, 3j, and 14a were equally effective against the chloroquine-sensitive D6 and the multi-drug resistant That strain, TM90-C235, while compound 3i had a two-fold higher IC₅₀ against TM90-C235 (D6 IC₅₀=957 ng/mL, 3 nM and TM90-C235 IC₅₀>2000 ng/ml,). The lack of cross-resistance in compounds 7a, 3j, and 14a is an encouraging finding for a novel scaffold and a valuable lead quality compound attribute given the rapid development of drug resistance against many antimalarials in the field. This initial finding is the basis for future research directed to the development of agents effective against P. falciparum.

TABLE 5
Inhibition of P. falciparum D6 and C235 by selected compounds.

| | D6 | | C235 | |
|---|---|---|---|---|
| | IC₅₀ | D6 | IC₅₀ | C235 |
| Compound | (ng/mL) | R² | (ng/mL) | IC₅₀ |
| Chloroquine | 3.8 | — | 46.1 | — |
| 3i | 956.9 | 0.93 | >2000 | 0.67 |
| 3j | 592.8 | 0.96 | 541 | 0.97 |
| 7a | 294.6 | 0.97 | 266.6 | 0.97 |
| 14a | 1331 | 0.95 | 1325 | 0.87 |

The in vitro data prompted the selection of 3i, 3j, 7a, 14a, and 14b for evaluation in mouse models of T. gondii infection. Initial difficulties were encountered with the formulation and preliminary safety studies of 3i, 3j, and 7a, presumably due to limited aqueous solubility. It was anticipated that 14a and 14b, by virtue of their polar, ionizable appended functionality, may possess improved physicochemical profiles. 14a and 14b were chosen for evaluation in a mouse model of T. gondii oocyst infection.

Todetermine whether 14a or 14b exerted toxic effects when dosed orally, each compound was administered by gavage to mice daily for nine days at a dose of 100 mg/kg. At the end of the ten days, all mice were alive and appeared healthy, suggesting that the neither compound had any observable toxic effect upon oral administration at the dosage studied.

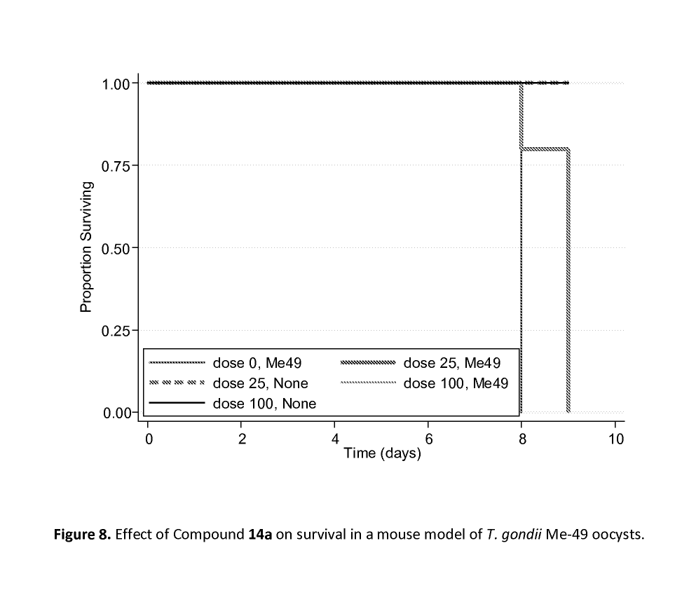

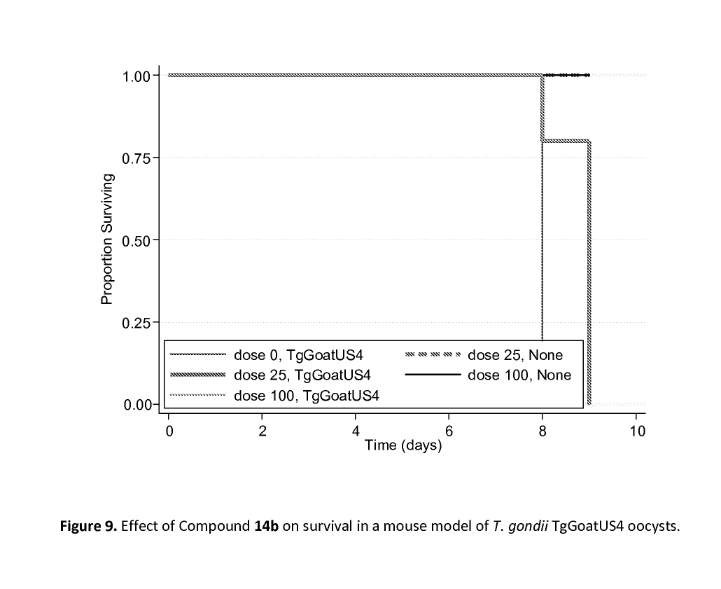

Thus, compounds 14a and 14b were tested for efficacy in a mouse model of T. gondii oocyst infection. The oocyst form of the parasite is excreted by cats and is often the form by which people and other animals become infected. This oocyst infection in mice is very virulent, and fatal. Mice were infected by oral gavage with ME49 or TgGoatUS4 oocysts. Mice were treated with either a high dose (100 mg/kg) or low dose (25 mg/kg) of 14a or 14b 1 mL suspension via oral gavage, or were not treated. All uninfected mice dosed with compound alone remained asymptomatic, whereas all mice inoculated orally with oocysts of either strain died of acute toxoplasmosis 8-9 days post infection, and tachyzoites were found in smears of their mesenteric lymph nodes. Treatment with 14a and 14b increased survival by 1 day (Table 6, FIGS. 8 and 9).

TABLE 6
Efficacy of compounds 14a and 14b in B7 mice infected
with T. gondii Me-49 or Tg-Goat-US4 oocysts.

| Compound | Dose | Challenge | # Mice | Day of Death |
|---|---|---|---|---|
| 14a | 100 mg/kg | None | 5 | None |
| 14a | 100 mg/kg | Me-49 | 5 | 8, 9, 9, 9, 9 |
| 14a | 25 mg/kg | None | 5 | None |
| 14a | 25 mg/kg | Me-49 | 5 | 8, 9, 9, 9, 9 |
| None | None | Me-49 | 5 | 8, 8, 8, 8, 8 |
| 14b | 100 mg/kg | None | 5 | None |
| 14b | 100 mg/kg | TgGoatUS4 | 5 | 8, 9, 9, 9, 9 |
| 14b | 25 mg/kg | None | 5 | None |
| 14b | 25 mg/kg | TgGoatUS4 | 5 | 8, 9, 9, 9, 9 |
| None | None | TgGoatUS4 | 5 | 8, 8, 8, 8, 8 |

Insertional mutagenesis experiments were performed. The goal of this study was the identification of one or more genes which, when disrupted, confer resistance to the parasite, thus potentially identifying the gene product which, upon interaction with the active compound, inhibits the growth of the parasite. THdhxgTRP tachyzoites were successfully transfected with pLK47 vector plasmid to create parasites with random gene mutations. No parasite growth was observed after prolonged incubation in the presence of 3i, 3j, 7a or 14b (Data not shown). The value of this approach to elucidate molecular targets or target pathways of T. gondii inhibitors was recently demonstrated. In an unrelated study conducted in our laboratory, this methodology has successfully identified the T. gondii trafficking pathway inhibited by a series of N-benzoyl-2-hydroxybenzamides. One interpretation of the data reported herein is that the molecular target of the active inhibitors of the study may be essential.

The initial in vitro screen yielded five promising agents, compounds 3i, 3j, 7a, 14a, and 14b which were active at low nanomolar concentrations and were not toxic to human host cells. Compound 3i had a static effect on parasite proliferation, which resumed after drug pressure was removed, while the activity of compounds 3j, 7a, and 14a was cidal. Compounds 14a and 14b compounds were effective at prolonging slightly the survival of mice infected with T. gondii oocysts, and showed no signs of toxicity. It will next be important to explore the activity of these compounds against the latent, encysted bradyzoite life stage.

3i, 3j, 7a, 14a were examined for activity against two drug-resistant strains of P. falciparum, the causative agent of malaria. All compounds demonstrated activity against P. falciparum, with 7a as the most effective.

Formulation of compounds of the invention may be prepared by those with skill in the art. It is well understood that components of formulations will differ according to the anticipated route of administration. It is anticipated that said components may include, but not be limited to, antiadherents, binders, coatings, disintegrants, dissolution modifiers, fillers, flavors, amphipathic agents, solubilizers, colors, lubricants, glidants, sorbents, preservatives, and sweeteners, the selection of which can be made by one with skill in the art, with such selection directed to one or more mode of administration. It is also anticipated that modification or optimization of the physical form of active compounds of the invention may be employed during the practice of administering said compounds to subjects in need of prevention or treatment of diseases caused by apicomplexan parasites. Such modifications are known by those with skill in the art and may include, but not be limited to, micronization, pulverization, process by nanotechnology, alternate crystal forms, hydrates, and resolution of optical isomers. It is further anticipated that compounds of the invention may be complexed, adhered, absorbed, or otherwise contacted to materials for the purpose of modifying pharmacokinetic properties. Such materials are known by those with skill in the art and may include, but not be limited to, peptides, proteins, lipozomes, phospholipids, polyethyleneglycols, detergents, surfactants, polysaccharides, cyclic ethers, and polymers.

Said formulation components, structure modifiers, and complexing agents are anticipated by the invention and are incorporated herein.

### EXAMPLES

### Synthesis of Potential Inhibitors

Unless otherwise stated, all solvents and reagents were used as received from vendors. ¹H NMR spectra were measured at either 400 MHz (Varian) or 500 MHz (Varian Inova AS500) in DMSO-d₆ or CDCl₃. HPLC-MS analyses were carried out with a Shimadzu LCMS 2020 using a Phenomenex CHO-8463 C18 column (50×3.0 mm) with a gradient of 10% acetonitrile: 90% water (0.1% formic acid) to 100% acetonitrile (0.1% formic acid) over five minutes. Retention times (T_{R}) are reported in minutes (min). Mass spectra (ESI) are reported in positive (m/z⁺) and/or negative (m/z⁻) mode. The calculated exact mass is denoted as EM. Unless otherwise stated, all compounds were obtained at >95% purity (HPLC-MS). Niclosamide (Compound 4) was purchased from Sigma-Aldrich (St. Louis, Mo.). Reactants and reagents were used as received from the vendor except as noted.

General Method of Salicylanilide Synthesis⁹.

A suspension of the salicylic acid derivative and aniline in xylenes (0.1 to 0.5 M) was warmed to reflux, and then a solution of phosphorous trichloride in CH₂Cl₂ (CAUTION: CH₂Cl₂ boils off rapidly at this temperature! Apparatus should be constructed to allow distillation of CH₂Cl₂!) or xylenes was introduced dropwise. When the reaction was complete, as determined by TLC or HPLC-MS, the reaction mixture was rapidly transferred while hot by pipette, cannulation, or decanting to a beaker and allowed to cool under rapid stirring. This action removed tarry residue which may accumulate on the reaction vessel walls during the reaction. Typically the product crystallized from the reaction solvent as it cooled, or was induced to crystallize upon the slow addition of hexanes when the temperature of the reaction solvent reached 75 to 80° C.

### Example 1

### N-(3-bromophenyl)-5-chloro-2-hydroxybenzamide (3b)

Using the method described for compound 3a, 5-chlorosalicylic acid (0.57 g, 3.30 mmol) reacted with 3-bromoaniline (0.36 mL, 3.30 mmol) and 2M PCl₃ in CH₂Cl₂ (0.66 mL, 1.32 mmol) in xylenes (8 mL). The crude product was recrystallized from EtOAc/hexanes. ¹H NMR (500 MHz, DMSO-d₆) δ 7.024 (m, 2H), 7.354 (m, 3H), 7.474 (m, 1H), 7.660 (m, 1H), 7.895 (m, 1H), 8.056 (s, 1H), 10.482 (s, 1H). HPLC T_{R} 2.84 min; m/z⁺ 327.85 [M+H]⁺; m/z⁻ 325.75 [M−H]⁻, (EM 324.95).

### Example 2

### N-(3-ethylphenyl)-5-chloro-2-hydroxybenzamide (3c)

Using the method described for compound 3a, 5-chlorosalicylic acid (0.63 g, 3.65 mmol) reacted with 3-ethylaniline (0.45 mL, 3.65 mmol) and 2M PCl₃ in CH₂Cl₂ (0.73 mL, 1.45 mmol) in xylenes (9 mL). The crude product was recrystallized from EtOAc/hexanes. ¹H NMR (400 MHz, DMSO-d₆) δ 1.198 (t, J=7.6 Hz, 3H), 2.618 (q, J=7.6 Hz, 2H), 7.012 (m, 2H) 7.254 (m, 1H), 7.457 (m, 3H), 7.983 (m, 1H), 10.357 (s, 1H). HPLC T_{R} 2.86 min; m/z⁺ 275.95 [M+H]⁺; m/z⁻ 273.80 [M−H]⁻; (EM 275.07).

### Example 3

### N-(3-(trifluoromethyl)phenyl)-5-chloro-2-hydroxybenzamide (3f)

Using the method described for compound 3a, 5-chlorosalicylic acid (2.19 g, 12.69 mmol) reacted with 3-trifluoromethylaniline (1.58 mL, 12.69 mmol) and 2M PCl₃ in CH₂Cl₂ (2.54 mL, 5.08 mmol) in xylenes (32 mL). The crude product was recrystallized from EtOH. ¹H NMR (500 MHz, DMSO-d₆) δ 7.035 (d, J=9.0 Hz, 1H), 7.488 (m, 2H), 7.619 (dd, J=8.0, 8.0 Hz, 1H), 7.934 (m, 2H), 8.209 (s, 1H), 10.624 (s, 1H). HPLC T_{R} 2.843 min; m/z⁺ 315.90 [M+H]⁺; m/z⁻ 628.80 [2M−H]⁻, 314.80 [M−H]⁻, (EM 315.03).

### Example 4

### N-(3-fluorophenyl)-5-chloro-2-hydroxybenzamide (3h)

Using the method described for compound 3a, 5-chlorosalicylic acid (2.42 g, 14.02 mmol) reacted with 3-amino benzonitrile (1.35 mL, 14.02 mmol) and 2M PCl₃ in CH₂Cl₂(2.80 mL, 5.61 mmol) in xylenes (30 mL). The crude product was recrystallized from 2-methyl-1-propanol. ¹H NMR (500 MHz, DMSO-d₆) δ 6.995 (m, 2H), 7.433 (m, 3H), 7.706 (m, 1H), 7.897 (d, J=2.8 Hz, 1H), 10.519 (s, 1H), 11.641 (s, 1H). HPLC T_{R} 2.639 min; m/z⁺ 265.90 [M+H]⁺; m/z⁻ 263.85 (EM 265.03).

### Example 5

### N-(3-tert-butylphenyl)-5-chloro-2-hydroxybenzamide (3i)

Using the method described for compound 3a, 5-chlorosalicylic acid (2.04 g, 11.82 mmol) reacted with 3-tert-butylaniline (1.76 g, 11.82 mmol) and 2M PCl₃ in CH₂Cl₂ (2.336 mL, 4.73 mmol) in xylenes (30 mL). At completion of reaction, the hot xylenes solvent was decanted, cooled to room temperature, and then diluted with hexanes (30 mL). This was stored at 4° C. for 30 hours during which time an off-white crystalline solid separated. The product was recrystallized from EtOAc/hexanes to give a mixture of the title compound (89.9% and an unidentified impurity (10.1%). ¹H NMR of the major component (400 MHz, DMSO-d₆) δ 1.280 (S, 9H), 7.172 (dq, J=8.0, 0.8 Hz, 1H), 7.283 (dd, J=8.0, 0.8 Hz, 1H), 7.455 (dd, J=8.8, 2.6 Hz⁻, 1H), 7.560 (dd, J=8.0, 0.8 Hz, 1H), 7.679 (M, 1H), 7.982 (d, J=2.6 Hz, 1H), 10.345 (S, 1H), 11.903 (S, 1H). HPLC T_{R} 3.095 min; m/z 303.95 [M+H]⁺; m/z⁻ =301.85[M−H]⁻; (EM 303.10).

### Example 6

### N-(3,5-bis(trifluoromethyl)phenyl-5-chloro-2-hydroxybenzamide (3j)

Using the method described for compound 3a, 5-chlorosalicylic acid (0.94 g, 5.48 mmol) reacted with 3,5-bis(trifluoromethyl)aniline (0.85 mL, 5.48 mmol) and 2M PCl₃ in CH₂Cl₂(1.10 mL, 2.19 mmol) in xylenes (15 mL). At completion of reaction, the hot xylenes solvent was decanted, cooled to room temperature, and then diluted with hexanes (50 mL). This was stirred at room temperature for 14 hours, during which time pure product separated as white crystals. ¹H NMR (400 MHz, DMSO-d₆) δ 7.048 (d, J=8.7 Hz, 1H), 7.493 (dd, J=9.0, 2.7 Hz, 1H), 7.845 (M, 2H), 8.449 (S, 2H), 10.851 (S, 1H), 11.427 (S, 1H). HPLC T_{R} 3.118 min; m/z⁻ 381.80; (EM 383.01).

### Example 7

### N-(3-benzyl phenyl)-5-chloro-2-hydroxybenzamide (3l)

Using the method described for compound 3a, 5-chlorosalicylic acid (0.81 g, 4.69 mmol) reacted with 3-benzylaniline (0.86 g, 4.69 mmol) and 2M PCl₃ in CH₂Cl₂(0.94 mL, 1.88 mmol) in xylenes (15 mL). The crude product was recrystallized from toluene. ¹H NMR (500 MHz, DMSO-d₆) δ 3.951 (S, 2H), 7.017 (m, 2H), 7.248 (m, 6H), 7.460 (dd, J=8.5, 2.5 Hz, 1H), 7.550 (m, 2H), 7.948 (d, J=2.0 Hz, 1H) 10.363 (s, 1H), 11.846 (s, 1H), HPLC T_{R} 3.017 min; m/z⁺ 337.95 [M+H]⁺; m/z⁻ 335.85; (EM 337.09).

### Example 8

### N-(2-chloro-5-nitrophenyl)-5-methyl-2-hydroxybenzamide (3m)

Using the method described for compound 3a, 5-methylsalicylic acid (0.77 g, 5.06 mmol) reacted with 2-chloro-4-nitroaniline (0.87 g, 5.06 mmol) and 2M PCl₃ in CH₂Cl₂ (1.01 mL0, 2.02 mmol)in xylenes (18 mL). The desired product (64.5% pure) was found to contain 5.5% of an unidentified contaminant after collection upon cooling of the reaction solvent. ¹H NMR (500 MHz, DMSO-d₆) δ 2.261 (s, 3H), 6.949 (d, J=8.5 Hz, 1H), 7.272 (dd, J=8.5, 2.0 Hz, 1H), 7.812 (d, J=2.0 Hz, 1H), 8.257 (dd, J=9.0, 2.5, 1H), 8.384 (d, J=2.5, 1H), 8.824 (d, J=9.0 Hz, 1H). HPLC T_{R} 2.628 min; m/z⁺ 306.95 [M+1]⁺; m/z⁻ 304.85 [M−H]⁻; (EM 306.04).

### Example 9

### N-(2,4-dichlorophenyl)-5-chloro-2-hydroxybenzamide (3n)

A suspension of 5-chlorosalicylic acid (1.73 g, 10.0 mmol) and 2,4-dichloraniline (1.62 g, 10.0 mmol) in xylenes (50 mL) was heated to reflux and a solution of PCl₃ (0.35 mL, 4.0 mmol) in xylenes (5.0 mL) was introduced in a dropwise manner. After 90 minutes, the reaction mixture was transferred to a beaker via pipette and was allowed to cool to rt under rapid stirring. The crude product was recrystallized from EtOAc. ¹H NMR (500 MHz, DMSO-d₆) δ 7.075 (dd, J=8.8, 1.2 Hz, 1H), 7.473 (m, 2H), 7.693 (dd, J=2.4, 1.2 Hz, 1H), 7.982 (dd, J=2.8, 1.2 Hz, 1H), 8.457 (dd, J=8.8, 1.2 Hz, 1H), 10.925 (s, 1H), 12.241 (s, 1H). HPLC T_{R} 2.889 min; m/z⁺ 315.85 [M+H]⁺; m/z⁻ 313.70 [M−H]⁻; (EM-314.96).

### Example 10

### 5-chloro-N-(4-chloro-2-fluorophenyl)-2-hydroxybenzamide (3p)

Using the method described for compound 3n, 5-chlorosalicylic acid (1.73 g, 10.0 mmol) and 2,4-difluoroaniline (1.46 g, 10.0 mmol) reacted in refluxing xylenes (25 mL) in the presence of PCl₃ (0.35 mL, 4.0 mmol). The crude product was recrystallized from EtOAc. ¹H NMR (500 MHz, DMSO-d₆) δ 7.057 (d, J=8.8 Hz, 1H), 7.335 (dd, J=8.8, 1.2 Hz), 7.508 (dd, J=8.8, 2.8 Hz, 1H), 7.577 (dd, J=10.4, 2.0 Hz, 1H), 7.959 (d, J=2.8 Hz, 1H), 8.245 (dd, J=8.8, 8.8 Hz), 10.704 (s, 1H), 12.166 (s, 1H). HPLC T_{R} 2.756 min; m/z⁺ 299.90 [M+H]⁺; m/z⁻ 297.75 [M−H]⁻; (EM=298.99).

### Example 11

### 5-chloro-N-(2-chloro-5-cyanophenyl)-2-hydroxybenzamide (3r)

Using the method described for compound 3n, 5-chlorosalicylic acid (0.86 g, 5.0 mmol) and 3-amino-4-chlorobenzonitrile (0.76 g, 5.0 mmol) reacted in refluxing xylenes (10 mL) in the presence of PCl₃ (0.18 mL, 2.0 mmol). The crude product was recrystallized from EtOH/water to provide a tan colored solid. ¹H NMR (500 MHz, DMSO-d₆) δ 7.053 (d, J=8.5 Hz), 7.345 (dd, J=8.5, 3.0 Hz), 7.407 (dd, J=8.5, 2.5 Hz), 7.598 (d, J=8.5 Hz), 8.079 (d, J=3.0 Hz), 8.979 (s, J=2.5 Hz), 11.105 (s, 1H), 11.700 (s, 1H.) HPLC T_{R} 2.551 min; m/z⁺ 306.90 [M+H]⁺; m/z⁻ =304.75 [M−H]⁻; (EM=306.00).

### Example 12

### 4-chloro-2-((2-chloro-4-nitrophenyl)carbamoyl)phenyl benzoate (7a)

Benzoyl chloride (0.49 mL, 4.27 mmol) was added dropwise to a suspension of niclosamide (1.27 g, 3.88 mmol) in a solution of 4-dimethylaminopyridine (DMAP, 30 mg) in pyridine (15 mL) at rt. The suspension was warmed to 80° C. whereupon all solids dissolved. Reaction continued at this temperature for 2 hr. The cooled reaction mixture was diluted with EtOAc (100 mL) and was washed successively with 1N HCl until the aqueous wash was acidic (about pH 1) to litmus. The EtOAc phase was washed with brine, dried (MgSO₄), and concentrated to an off-white solid. The crude product was recrystallized from EtOAc/hexanes. ¹H NMR (500 MHz, DMSO-d₆) δ 10.56 (s, 1H), 8.31 (m, 1H), 8.25 (m, 1H), 8.08 (m, 2H), 7.90 (m, 2H), 7.25 (m, 1H), 7.54 (m, 3H).). HPLC T_{R} 2.990 min; m/z⁺ 430.95 [M+H]⁺; m/z⁻ 428.80 [M−H]⁻; (EM 430.01).

### Example 13

### 4-chloro-2-((2-chloro-4-nitrophenyl)carbamoyl)phenyl 4-methylpiperazine-1-carboxylate (7d)

4-Methyl-1-piperazinecarbonyl chloride (0.603 g, 3.02 mmol) was added to a suspension of niclosamide 4 (0.495 g, 1.51 mmol) in pyridine (8.0 mL) containing DMAP (10 mg). The mixture was raised to reflux for 1 hr. The hot solution was introduced by pipette to rapidly stirring water at rt. After 1 hr, the solids were collected by filtration and added to 50 mL rapidly stirring 1N HCl. This was stirred for 30 min, then the crude product was collected by filtration and dried in a vacuum oven (28″ Hg, 50° C.) for 18 hr, then recrystallized from EtOH. A small portion of the product was converted to the free base by partitioning between EtOAc and saturated NaHCO₃. TLC (SiO₂, 5 MeOH: 95 CHCl₃) demonstrated a single component, Rf=0.37. ¹H NMR (400 MHz, CDCl₃) δ 8.91 (s, 1H), 8.81 (d, J=9.2 Hz), 8.34 (d, J=2.4 Hz), 8.22 (dd, J=9.2, 2.4 Hz), 7.87 (d, J=2.8 Hz), 7.51 (dd, J=2.8, 8.4 Hz), 7.13 (d, J=8.4 Hz), 3.68 (m, 2H), 3.56 (m, 2H), 2.39 (m, 4H), 2.28 (s, 3H). HPLC T_{R} 1.664; m/z⁺ 452.95 [M+H]⁺; m/z⁻ 450.90 [M−H]⁻; (EM 452.07).

### Example 14

### tert-butyl 2-((chlorocarbonyl)(methyl)amino)acetate (12)

A solution of sarcosine tert-butyl ester hydrochloride 11 (4.214 g, 23.20 mmol) in CH₂Cl₂ (40 mL) was shaken with saturated NaHCO₃ in a separatory funnel. The organic phase was dried over MgSO₄ and concentrated to a clear oil (2.298 g, 68% yield). A solution of phosgene in toluene (20%, 10.9 mL, 23.75 mmol) was cooled to −25° C., and a solution of sarcosine tert-butyl ester (2.298 g, 15.78 mmol) and DIEA (5.5 mL, 31.66 mmol) in CH₂Cl₂ (10 mL) was introduced in a dropwise fashion. The solution was allowed to warm to rt over 1 hr, and was then washed with 1N HCl (50 mL) and EtOAc sufficient to form two layers was added. The organic phase was washed with water, then brine, and dried over MgSO₄. This solution was used without further manipulation.

### Example 15

### 2-(((4-chloro-2-((2-chloro-4-nitrophenyl)carbamoyl)phenoxy)carbonyl)(methyl)amino)acetic acid (13a)

A. tert-Butyl 2-(((4-chloro-2-((2-chloro-4-nitrophenyl)carbamoyl)phenoxy)carbonyl)(methyl)amino)acetate. A solution of 12 (15.78 mmol) in EtOAc (ca. 0.5M) was introduced dropwise to a refluxing solution of niclosamide (2.56 g, 7.92 mmol) in pyridine (80 mL containing DMAP (50 mg). After 30 min, 100 mL solvent was distilled from the reaction mixture. The remaining reaction mixture was cooled to rt, diluted with EtOAc (50 mL), and washed successively with 1N HCl until the aqueous wash was acidic (about pH 1) to litmus. The organic phase was washed with brine, dried (MgSO₄), and concentrated to an off-white solid. This was used without further purification. B. 2-(((4-chloro-2-((2-chloro-4-nitrophenyl) carbamoyl)phenoxy)carbonyl) (methyl)amino) acetic acid. The solid from Step A (542 mg, 1.09 mmol) was dissolved in CH₂Cl₂ (15 mL) and CF₃COOH (15 mL) was added. After 16 hr the reaction solution was concentrated to an oily residue. This was dissolved in CHCl₃ (25 mL) and concentrated. The CHCl₃ chase was repeated to leave a scinterable foam. This was layered with 35 EtOAc: 65 hexanes (50 mL), and warmed to 45° C. under rapid stirring for 30 min. Hexanes (30 mL) was added, and the stirring mixture was allowed to cool to rt over 20 min. The product was collected by filtration. The ¹H NMR spectrum was complex and revealed the existence of rotational isomers, presumably due to restricted rotation about the sarcosine-1-carbamoyl bond. Analysis of HPLC and MS data reveals a single compound in ≧95% purity. T_{R} 2.382 min; m/z⁺ 441.85 [M+H]⁺; m/z⁻ 324.75 [M-sarcosine-H]; (EM 441.01).

### Example 16

### 4-chloro-2-((2-chloro-4-nitrophenyl)carbamoyl)phenyl (2-hydrazinyl-2-oxoethyl)(methyl)carbamate hydrochloride (13b)

A. Tert-butyl 2-(2-(((4-chloro-2-((2-chloro-4-nitrophenyl)carbamoyl)phenoxy)carbonyl)(methyl)amino)acetyl)hydrazinecarboxylate. Tert-butyl carbazate (417 mg, 3.16 mmol) was added to a solution of 13a (1.27 g, 2.87 mmol) in THF (8.7 mL) with stirring at rt. A solution of N-(3-dimethylaminopropyl)-N′-ethylcarbodiimide hydrochloride (660 mg, 3.44 mmol) in CH₂Cl₂ (17.4 mL) was introduced dropwise at rt. The homogeneous solution was stirred at rt for 14 hr, then concentrated. The residue was partitioned between EtOAc and 1 N HCl. The EtOAc phase was washed with water, then with brine, dried (MgSO₄), and concentrated to an off-white solid 1.20 g (75.1%). This was used without further manipulation. B. 4-Chloro-2-((2-chloro-4-nitrophenyl)carbamoyl)phenyl (2-hydrazinyl-2-oxoethyl)(methyl)carbamate hydrochloride. The product from step A (2.52 g, 5.70 mmol) was dissolved in a solution of HCl in dioxane (4.0 M, 10 mL) and the solution was stirred at rt for 30 min, then concentrated to an off-white solid which was washed well with EtOAc, then hexanes, and dried under a stream of air to provide a white solid. (2.50 g, 89%). ¹H NMR spectrum was complex and revealed the existence of two rotational isomers, presumably due to restricted rotation about the sarcosine-1-carbamoyl bond. ¹H NMR (500 MHz, DMSO-d₆) δ 11.09 (s, 1H), 10.45 (s, 1H), 10.40 (s, 1H), 8.41 (dd, J=2.3, 1.5 Hz, 1H), 8.31-8.25 (m, 1H), 8.10 (m, 1H), 7.80 (dd, J=6.2, 2.6 Hz, 1H), 7.71-7.65 (m, 1H), 7.36 (d, J=8.7 Hz, 1H), 7.26 (d, J=8.7 Hz, 1H), 4.21 (s, 1H), 4.05 (s, 1H), 3.07 (s, 1.5H), 2.92 (s, 1.5H). HPLC T_{R} 2.03 min; m/z⁺ 455.95 [M+H]⁺; m/z⁻ 453.95 [M−H]⁻; (EM 308.06).

### Example 17

### 2-(((2-((2,4-bis(trifluoromethyl)phenyl)carbamoyl)-4-chlorophenoxy)carbonyl)(methyl)amino)acetic acid (14a)

A. tert-Butyl 2-(((2-((2,4-bis(trifluoromethyl)phenyl)carbamoyl)-4-chlorophenoxy)carbonyl)(methyl)amino)acetate. N-(3,5-bis(trifluoromethyl)phenyl-5-chloro-2-hydroxybenzamide (3j, 6.47 g, 16.86 mmol) was added to a solution of 12 (4.20 g, 20.22 mmol) in pyridine (35 mL) and the mixture was warmed to 80° C. for three hours. The reaction solution was cooled to rt, diluted with EtOAc (200 mL) and washed four times with 1N HCl (final wash ph about 1 to litmus), once with brine, and dried (MgSO₄). Concentration afforded an off-white solid which was triturated with hexanes to provide the product (7.01 g, 75%) which was used without further purification. B. 2-(((2-((2,4-bis(trifluoromethyl)phenyl)carbamoyl)-4-chlorophenoxy)carbonyl)(methyl)amino)acetic acid. The product from step A (5.43 g, 9.70 mmol) was dissolved in CH₂Cl₂ (15 mL) and CF₃COOH (7.5 mL) was added. The reaction mixture was stirred at rt for 14 hr, then the reaction mixture was concentrated and the residue was chased twice with CHCl₃ (25 mL).

The ¹H NMR spectrum was complex and revealed the existence of rotational isomers, presumably due to restricted rotation about the sarcosine-1-carbamoyl bond. Analysis of HPLC (T_{R}=2.65 min) and MS data (m/z⁺ 499.05, [M+H]⁺, m/z⁻ 381.80, [M-C4H6NO]⁻) reveals a single compound in ≧95% purity.

### Example 18

### 2-((2,4-bis(trifluoromethyl)phenyl)carbamoyl)-4-chlorophenyl (2-hydrazinyl-2-oxoethyl)(methyl)carbamate hydrochloride (14b)

A. tert-Butyl 2-(2-(((2-((2,4-bis(trifluoromethyl)phenyl)carbamoyl)-4-chlorophenoxy)carbonyl)(methyl)amino)acetyl)hydrazine carboxylate. A solution of 2-(((2-((2,4-bis(trifluoromethyl)phenyl)carbamoyl)-4-chlorophenoxy)carbonyl)(methyl)amino)acetic acid (14a, 540 mg, 1.08 mmol) in CH₂Cl₂ (5.0 mL0 was treated with tert-butyl carbazate (172 mg, 1.30 mmol) and N-(3-dimethylaminopropyl)-N′-ethylcarbodiimide hydrochloride (249 mg, 1.30 mmol). The solution was stirred at rt for 4 hr, then concentrated, and the residue was dissolved in EtOAc (15 mL), washed with 1N HCl, then with brine, dried over MgSO₄, and concentrated to a scinterable foam.). The ¹H NMR spectrum was complex and revealed the existence of rotational isomers, presumably due to restricted rotation about the sarcosine-1-carbamoyl bond. Analysis of HPLC (T_{R}=2.81 min) and MS data (m/z⁺ 634.90 [M+Na]⁺, m/z⁻ 610.85, [M−H]⁻) reveals a single compound in ≧95% purity. B. 2-((2,4-bis(trifluoromethyl)phenyl)carbamoyl)-4-chlorophenyl (2-hydrazinyl-2-oxoethyl)(methyl)carbamate hydrochloride (14b, 2.263 g, 3.69 mmol) was dissolved in a solution of HCl in dioxane (15 mL). After stirring 2 hr at rt, the reaction mixture was concentrated and the residue was chased twice with 15 mL portions of CHCl₃, providing a scinterable foam (1.90 g, 94%). ¹H NMR (500 MHz, DMSO-d₆) δ 11.03 (s, 1H), 10.95 (s, 1H), 9.15 (s, 1H), 9.08 (s, 1H), 8.36 (s, 4H), 7.84 (s, 2H), 7.80 (dd, J=5.2, 2.7 Hz, 2H), 7.70-7.64 (m, 1H), 7.38 (d, J=8.7 Hz, 1H), 7.26 (d, J=8.7 Hz, 1H), 4.17 (q, J=18.2, 13.8 Hz, 3H), 3.97 (s, 2H), 3.79 (s, 2H), 3.00 (s, 3H), 2.83 (s, 3H). HPLC T_{R} 2.38 min; m/z⁺ 512.90 [M+H]⁺; m/z⁻ 510.80 [M−H]⁻; (EM 512.07).

### Example 19

### In Vitro Evaluation of Inhibition of T. Gondii Tachyzoites

Test compounds were dissolved in DMSO to make a 10 mM solution, and subsequently diluted with IMDM-C to the concentrations used in bioassay. In the in vitro experiments DMSO concentration was not greater than 0.1% unless otherwise specified. RH-YFP parasites, which stably express Yellow Florescent Protein, were used. Tachyzoites were extracted from HFF cells by double passage through a 25-gauge needle, centrifuged for 15 minutes at 1500 RPM, and resuspended in IMDM-C. Confluent monolayers of HFF cells were infected with parasites in 96-well plates (Falcon 96 Optilux Flat-bottom) with 3,500 parasites in 100 uL per well. One hour after inoculation, test compounds and control media were added for a final volume of 200 uL per well. Parasite proliferation was assessed using [³H]-Uracil incorporation or YFP Fluorescence assay.

T. gondii Parasite and Cell Culture.

Human Foreskin Fibroblast (HFF) cells were maintained in confluent monolayers in Iscoves's Modified Dulbecco's Medium supplemented with 10% fetal bovine serum, 1% GlutaMAX and 1% penicillin-streptomycin-fungizone (IMDM-C). T. gondii tachyzoites were cultivated in HFF monolayers. Parasites and cells were maintained at 33° C. or 37° C. and 5% CO₂. The strains of parasite used in this study include RH, RH-YFP, and Prugnaud FLUC (Type 2 parasites stably transfected with luciferase), Me49 strain, and TgGoatUS4 isolate.

### Example 20

### [³H]-Uracil and [³H]-Thymidine incorporation assays

25 μL of 0.1 mCi/mL*³H]-Uracil or [³H]-Thymidine was added to each well 24 hours before reading plates. At harvesting, contents of the wells were transferred onto a 96-well UniFilter GF/C filter plates using a Filtermate 196 cell harvester (Packard). [³H]-Uracil and [³H]-Thymidine incorporation was measured using a Microplate Scintillation Luminescence Counter (Packard).

### Example 21

### YFP Florescence Assay

72 hours after initiation of the in vitro challenge assay, parasite proliferation was assessed by reading fluorescence of YFP parasites with a Synergy H4 Hybrid Reader (BioTek) and Gen5 1.10 software, using a bottom optics positions, excitation wavelength of 514 nm, and emission wavelength of 540 nm.

### Example 22

### In Vitro Toxicity Assay

HFF cells were grown to 30% confluence in 96-well plates.

Inhibitory compounds and control media were added to wells in concentrations equal to those being tested in challenge assays. After 72 hours, [³H]-Thymidine incorporation assay was conducted to assess cell growth. Alternatively, toxicity was assessed using WST-1 cell proliferation reagent (Roche). Confluent HFF cells were treated with inhibitory and control compounds. On the final day of experiment, 10 uL of WST-1 reagent was added to each well. Plates were incubated for 1 hour in the dark at 37° C., and absorbance was measured using Synergy H4 Hybrid Reader (BioTek) fluorometer at 420 nM.

### Example 23

### In Vivo FLUC Challenge Assay

Mice were infected intraperitoneally with 20,000 FLUC parasites in 400 uL of PBS on day one. One hour later, mice were injected with compound at various concentrations, dissolved in 100 uL of DMSO or DMSO control. The compound was administered daily for 6 days. Parasite burden was assessed by daily imaging with a Xenogen camera. This experiment was initiated with 4 or 5 mice per group.

### Example 24

### Anti-Plasmodial SYBR Green 1-Based Fluorescence (MSF) Assay

D6 (CDC/Sierra Leone) and TM90-C235 (WRAIR, Thailand) laboratory strains of P. falciparum were used for each drug sensitivity assessment. The parasite strains were maintained continuously in long-term cultures as previously described in Johnson et al, and P. falciparum strains in late-ring or early-trophozoite stages were cultured in predosed 384-well microtiter drug assay plates in 38 μl culture volume per well at a starting parasitemia of 0.3% and a hematocrit of 2%. Pre-dosed, sterile, 384 well black optical bottom microtiter drug plates for use in the MSF assay were produced using a Tecan EVO Freedom Liquid Handling System (Tecan US, Durham, N.C.). Dose response plates were produced at a final concentration ranging from 0.5-10000 ng/ml in quadruplicate (twelve two-fold serial dilutions of each test compound or chloroquine control in DMSO. Each run was validated by a batch control plate with chloroquine (Sigma-Aldrich Co., Catalog #C6628) at a final concentration of 2000 ng/ml. The cultures were incubated for 72 hours at 37° C., 5% CO2, 5% O2 and 90% N2. Lysis buffer (38 μl per well), consisting of 20 mM Tris HCl, 5 mM EDTA, 1.6% Triton X, 0.016% saponin, and SYBR green I dye at a 20× concentration (Invitrogen, Catalog #S-7567) was then added to the assay plates utilizing the Tecan EVO Freedom system for a final SYBR Green concentration of 10×. Plates were incubated in the dark at room temperature in the dark for 24 hours.

Compound activity was assessed by examining for the relative fluorescence units (RFU) per well using the Tecan Genios Plus (Tecan US, Inc., Durham, N.C.). GraphPad Prism (GraphPad Software Inc., San Diego, Calif.) using the nonlinear regression (sigmoidal dose-response/variable slope) equation was used to determine IC₅₀ values.

### Example 25

### Determination of Static or Cidal Effects

RH-YFP tachyzoites were treated with each compound at 1 μM under one of four conditions: a) parasites were treated for four days, then compound was removed; b) parasites were treated for ten days, then compound was removed; c) compound was refreshed at four days and removed at ten; or d) compound was maintained for the duration of the experiment. The four and ten day time points were taken to reveal the impact of extended exposure of the parasites to the test substance. Compounds were refreshed at four days to examine whether compound degradation could contribute to an observed static effect. Parasite growth was assessed at 11, 17, and 25 days.

### Example 26

### In Vivo Toxicity and Oocyst Assays

HLA B07 transgenic mice were produced at Pharmexa-Epimmune (San Diego, Calif., USA) and bred at the University of Chicago. All studies were conducted with Institutional Animal Care and Use Committee at the USDA, the University of Chicago, and the University of Strathclyde.

### Example 27

### Infection of Mice with T. Gondii Oocysts

Oocysts were obtained by feeding infected tissues of Swiss Webster mice to cats, sporulated in 2% sulfuric acid on a shaker for one week, and stored at 4° C. until used (Dubey 2010). Oocysts were counted in a disposable hemocytometer and diluted 10-fold from 10⁻¹ to 10⁻⁷ to reach an end point of ≅1 oocyst. All ten-fold dilutions were made in 50 ml tubes with 2% sulfuric acid (5 ml aliquot+45 ml sulfuric acid), and dilutions were stored at 4° C., to avoid variability in inocula preparations. For inoculation of mice, oocysts from the designated dilution were neutralized with 3.3% sodium hydroxide with neutral red as indicator (approximately the same volume as the inoculum). The resultant mixture was inoculated orally into 5 mice for each dilution (unless indicated otherwise) via a gastric needle with a blunt bulb (22 gauge, 50 mm long, Cadence Science catalogue no. 7920), without washing to avoid variability of the inocula during washing procedure. All orally inoculated mice were housed in autoclavable rodent cages with biohazard signs to incinerate bedding and food for 10 days to avoid spread of T. gondii because some oocysts pass unencysted in mouse feces.

### Example 27

### Bioassay of T. gondii in Mice

Mice were observed daily for the duration of the experiment. All mice were examined for T. gondii infection. Impression smears of tissues (usually mesenteric lymph nodes or lungs) were examined microscopically for tachyzoites. Survivors were bled six to eight weeks later and 1:25 dilution of their sera were examined for T. gondii antibodies using the modified agglutination test. The last infective dilution was considered to have 1 viable organism. The inoculated mice were considered infected with T. gondii when tachyzoites or tissue cysts were found in tissues. Seroconversion at 6 weeks was considered as indication of the presence of live parasites in the inocula. However, brains of all mice that survived 6 weeks were examined for tissue cysts, irrespective of serological results. With the strains of T. gondii used here, tissue cysts are found in all seropositive mice.

Evaluation of Efficacy of 14a in a Mouse Model of T. gondii Oocyst Infection

Five groups of B7 female mice weighing approximately 25 g were used. Group 1 received only 14a (100 mg/kg, 2.5 mg/mL, 1.0 mL) by oral gavage. Group 2 received only 14a (25 mg/kg, 0.5 mg/mL, 1.0 mL) by oral gavage. Group 3 received 14a (100 mg/kg, 2.5 mg/mL, 1.0 mL) and 100 ME49 oocysts by oral gavage. Group 4 received 14a (25 mg/kg, 0.5 mg/mL, 1.0 mL) and 100 ME49 oocysts by oral gavage. Group 5 received only 100 ME49 oocysts by oral gavage.

### Example 28

### Evaluation of Efficacy of 14b in a Mouse Model of T. gondii Oocyst Infection

Five groups of B7 female mice weighing approximately 25 g were used. Group 1 received only 14b (100 mg/kg, 2.5 mg/mL, 1.0 mL) by oral gavage. Group 2 received only 14b (25 mg/kg, 0.5 mg/mL, 1.0 mL) by oral gavage. Group 3 received 14b (100 mg/kg, 2.5 mg/mL, 1.0 mL) and 100 TgGoatUS4 oocysts by oral gavage. Group 4 received 14a (25 mg/kg, 0.5 mg/mL, 1.0 mL) and 100 TgGoatUS4 oocysts by oral gavage. Group 5 received only 100 TgGoatUS4 oocysts by oral gavage.

### Example 29

### Insertional Mutagenesis Experiments

THdhxgTRP tachyzoites were transfected with pLK47 vector plasmid. Parasites were extracted from HFF cells and resuspended in 1 mL cytomix electroporation buffer solution (120 mM KCl, 150 uM CaCl₂, 5 mM K₂HPO₄, 5 mM KH₂PO₄, 25 mM HEPES, 2 mM EDTA and 5 mM MgCl₂ in sterile H₂O). Equivalent amounts of plasmid DNA and cytomix solution containing 10×10⁷ parasites were combined for a total volume of 400 uL and electroporated using BioRad electroporator at 1.5 k, 25 fF and 100Ω. This resulted in a random insertion of the plasmid in the parasite genome and random disruption of a gene. Successfully transfected parasites were selected by month-long treatment with mycophenolic acid (25 ug/mL) and xanthine (50 ug/mL). After one month, the mixed mutant population were exposed to 3i, 3j, 7a or 14b to select for those mutants whose gene disruption conferred resistance to the drug. No parasite growth was noted after six weeks of exposure to mycophenolic acid, xanthine and any of the four test compounds.

All citations are incorporated by reference.

1. The method of use of one or more compound of Formula I to prevent or treat a disorder caused wholly or in part by one or more apicomplexan parasite, wherein said method comprises contacting an animal in need of said prevention or treatment with an effective amount said compound, wherein said compound has the structure 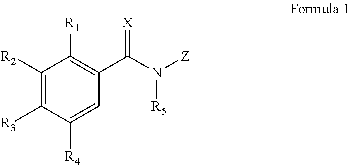
wherein
R₁═H, SH, or OR₆;
R₂ and R₃ are selected from fused phenyl, H, lower alkyl, I, Br, Cl, F, CF₃, CH₂CF₃, CH₂Ph, CH═CH₂, C≡CH, OCH₃, OCF₃, Ph, OPh, and NO₂;
R₄ is selected from the group consisting of H, lower alkyl, I, Br, Cl, F, CF₃, CH₂CF₃, CH₂Ph, CH═CH₂, C≡CH, C≡N, OCH₃, OCF₃, Ph, OPh, and NO₂;
R₅ is H, lower alkyl, or phenyl;
R₆ is selected from the group consisting of H, COCH₃, COCH₂CH₃, COCH(CH₃)₂, COC(CH₃)₃, COPh, COCH₂Ph, COC₆H₄NO₂(p), COC₆H₄OH(p), COC₆H₄NH₂(p), CON(CH₃)₂, CON(CH₂CH₃)₂, CON(CH₃)(CH₂CH₃), CON(CH₂Ph)₂, CON(CH₃)(CH₂Ph), 1-pyrrolidinecarbonyl, 2-carboxy-1-pyrrolidinecarbonyl, (2S)-2-carboxy-1-pyrrolidinecarbonyl, (2R)-2-carboxy-1-pyrrolidinecarbonyl, 1-morpholinecarbonyl, 4-methyl-1-piperazinecarbonyl, sarcosine-N-carbonyl, CO-N-Me-Ala-OH, CO-N-Me-Val-OH, CO-N-Me-Leu-OH, CO-N-Me-Ile-OH, CO-N-Me-Val-OH, CO-N-Me-Met-OH, CO-N-Me-Phe-OH, CO-N-Me-Trp-OH, CO-Pro-OH, CO-N-Me-Gly-OH, CO-N-Me-Ser-OH, CO-N-Me-Thr-OH, CO-N-Me-Cys-OH, CO-N-Me-Tyr-OH, CO-N-Me-Asn-OH, CO-N-Me-Gln-OH, CO-N-Me-Asp-OH, CO-N-Me-Glu-OH, CO-N-Me-Lys-OH, CO-N-Me-Arg-OH, CO-N-Me-His-OH, CO-N-Me-Gly-Gly-OH, CO-N-Me-Gly-Gly-Gly-OH, CO-N-Me-Gly-Phe-OH, CO-N-Me-Gly-Glu-OH, CO-N-Me-Gly-Glu-Glu-OH, CO-N-Me-Glu-Glu-OH, CO-N-Me-Gly-Lys-OH, CO-N-Me-Gly-Lys-Lys-OH, CO-Pro-Glu-OH, CO-Pro-Glu-Glu-OH, CO-Pro-Gly-OH, CO-Pro-Gly-Lys-OH, and CO-Pro-Lys-Lys-OH;
X is O or S; and
Z is substituted phenyl, or substituted 5- or 6-membered heterocyclic ring containing 1 or 2 heteroatoms chosen from N, O, and S, wherein said phenyl substituents are selected from the group consisting of H, lower alkyl, I, Br, Cl, F, CF₃, CH₂CF₃, CH₂Ph, CHPh₂, CH═CH₂, E-CH═CHCH₃, Z—CH═CHCH₃, C≡CH, C≡N, OCH₃, OCF₃, OPh, and NO₂ and wherein said heterocyclic ring substituents are selected from the group consisting of H, lower alkyl, I, Br, Cl, F, CF₃, CH₂CF₃, CH₂Ph, CH═CH₂, C≡CH, C≡N, OCH₃, OCF₃, Ph, OPh, and NO₂.
